SEQUENCE LISTING (57 sequences)

SEQ ID NO: 1 - protein, 221 aa, Mus musculus
MGTVVAKLLLPTLSSLAFLPTVSIATKRRFYMEAMVYLFTMFFVAFSHACDGPGLSVLCFMRRDILEYFSIYGTALSMWVSLMALADFDEPQRSTFTMLGVLTIAVRTFHDRWGYGVYSGPIGTATLIIAVKWLKKMKEKKGLYPDKSIYTQQIGPGLCFGALALMLRFFFEEWDYTYVHSFYHCALAMSFVLLLPKVNKKAGNAGAPAKLTFSTLCCTCV

SEQ ID NO: 2 - DNA, 666 bp, Mus musculus
atggggacagttgtagccaaactgctcctgcctaccctcagcagcctggccttcctcccgacagtgagcatcgctaccaagaggcgtttctacatggaggccatggtctacctcttcaccatgttctttgtggcgttctcccatgcctgtgatgggcctggtttgtctgtgctgtgcttcatgcgccgtgacattctggagtacttcagcatctatggaacagccctgagcatgtgggtctccctgatggcactggccgactttgatgaaccccagagatcgaccttcacaatgcttggcgtccttaccatcgctgtgcggacttttcatgaccgctggggttacggggtatactccggtcccataggcacggccaccctcatcattgctgtaaagtggctgaagaagatgaaagagaagaagggcctgtaccccgacaagagcatctacacccagcagataggccccggcctgtgctttggggccctggccctgatgcttcgattcttctttgaggaatgggattacacctacgtccacagcttctaccactgtgccctggccatgtcctttgtcctgctgctgcccaaggtcaacaagaaggctgggaacgcaggggcccccgccaagctgaccttctccaccctctgctgcacttgtgtctga

SEQ ID NO: 3 - protein, 221 aa, Homo sapiens
MGTLVAKLLLPTLSSLAFLPTVSIAAKRRFHMEAMVYLFTLFFVALHHACNGPGLSVLCFMRHDILEYFSVYGTALSMWVSLMALADFDEPKRSTFVMFGVLTIAVRIYHDRWGYGVYSGPIGTAILIIAAKWLQKMKEKKGLYPDKSVYTQQIGPGLCFGALALMLRFFFEDWDYTYVHSFYHCALAMSFVLLLPKVNKKAGSPGTPAKLDCSTLCCACV

SEQ ID NO: 4 - DNA, 666 bp, Homo sapiens
atggggacgctggtggccaagctgctcctgcccaccctcagcagcctggccttcctccccactgtcagcatcgcggccaagaggcggttccacatggaggccatggtctacctcttcaccctgttcttcgtggcgctccaccatgcctgcaatggacccggcttgtctgtgctgtgcttcatgcgtcacgacatcctggagtatttcagtgtctacgggacagccctgagcatgtgggtctcgctgatggcactggccgacttcgacgaacccaagaggtcaacatttgtgatgttcggcgtcctgaccattgctgtgcggatctaccatgaccgatggggctacggggtgtactcgggccccatcggcacagccatcctcatcatcgcggcaaagtggctacagaagatgaaggagaagaagggcctgtacccagacaagagcgtctacacccagcagataggccccggcctctgcttcggggcgctggccctgatgctacgcttcttctttgaggactgggactacacttatgtccacagcttctaccactgtgccctggctatgtcctttgttctgctgctgcccaaggtcaacaagaaggctggatccccggggaccccggccaagctggactgctccaccctgtgctgtgcttgtgtctga

SEQ ID NO: 5 - protein, 84 aa, Mus musculus
MPVPLLPMVLRSLLSRLLLPVARLARQHLLPLLRRLARRLSSQDMREALLSCLLFVLSQQQPPDSGEASRVDHSQRKERLGPQK

SEQ ID NO: 6 - DNA, 255 bp, Mus musculus
atgcccgttccattgctcccgatggtgcttcgatcgctgctgtcccgcctgctgctgcctgttgcccgcctggcccggcagcacctcctgcccttgctgcgccggctggcccgccgactgagctcccaagacatgagagaggctctgctgagctgtctgctctttgtcctcagccagcaacagccaccggattctggagaggcctccagagtggaccactcccagaggaaggagagattgggcccccagaagtga

SEQ ID NO: 7 - protein, 84 aa, Homo sapiens
MPTPLLPLLLRLLLSCLLLPAARLARQYLLPLLRRLARRLGSQDMREALLGCLLFILSQRHSPDAGEASRVDRLERRERLGPQK

SEQ ID NO: 8 - DNA, 255 bp, Homo sapiens
atgcccacgccactgctcccgctgctgcttcgattgctgctgtcctgcctgctgctgcctgctgcccgcctggcccgccaatacctcctgcccctgctgcgccgattggcccgccgcctgggctcccaggacatgcgagaggctttgctgggctgtctgctgttcattctcagccagcgacactcgccagacgctggggaggcctcaagagtggaccgcctggagaggagggagaggttaggcccccaaaagtga

SEQ ID NO: 9 - protein, 84 aa, Homo sapiens; identical to SEQ ID NO: 7

SEQ ID NO: 10 - protein, 84 aa, Mus musculus; identical to SEQ ID NO: 5

SEQ ID NO: 11 - protein, 84 aa, Rattus rattus
MPVPLLPLMLRSLLSRLLLPVARLARQHLLPLLRRLARRLSSQDVREALLSCLLFVLSQQQPPDSGETSRVDHSQRKERLGPRK

SEQ ID NO: 12 - protein, 85 aa, Oryctolagus cuniculus
MPAPLLPLLLRTLLSRLLLPAARLARRHLLPLLRRLAQRLGSQGTREALLGCLLFVLSQRQPPDASGEASRVDPPERKERLGRQK

SEQ ID NO: 13 - protein, 84 aa, Capra hircus
MPAPLLPLLLRTLLARLLLPAARLARQHLLPLLRRLARRLGSQDLREALLGCLLFVLSQRRPPDAGEASRVARLERRERLASQK

SEQ ID NO: 14 - protein, 84 aa, Delphinus delphis
MPTLLLPLLLRTLLARLLLPAARLARRHFLPLLRRLVHRLGSQDMREALLGCLLFVLSQRHPPDAGEASRVARLERRDRLASQK

SEQ ID NO: 15 - protein, 85 aa, Camelus dromedarius
MPAPLLPLLLLRTLLARLLMPAVRLARRHLLPLLRRLARRLGSQDMREALLGCLLFILSQRHQPDAGEPSKVARLERRERLAPQK

SEQ ID NO: 16 - protein, 84 aa, Panthera pardus
MPAPLLPLLLRTLMSRLLLPATRLARRHLLPLLRRLARRLGSQDVREALLGCLLFILSQSRPPDAEEVSRVAGQERRERLAPPK

SEQ ID NO: 17 - protein, 84 aa, Manis javanica
MPVPLLPMLVRTLLARLLLPAARLARQHLLPLLRRLAQRLGSHDMREALLGCLLFVLSQRHPPDAEEASRVARPERRGRLSPQK

SEQ ID NO: 18 - protein, 84 aa, Ailuropoda melanoleuca
MPAPLLPLLLRTLMSRLLLPVARMARRHLLPLLCRLARRLGSQDVREALLGCVLFVLSQRRPPDAEVASRVAGQERRERLAPPK

SEQ ID NO: 19 - protein, 84 aa, Desmodus rotundus
MPAPLLPLLLRMLLSRLLLPAARLARQHLLPLLRRLARRLGSQDLREALLSCLLFILSQRHQPDTGEASRVARPERRERLAPQK

SEQ ID NO: 20 - protein, 84 aa, Loxodonta africana
MPVPLLSLLLRALLSRLLLPAARLARQHLLPLLRRLARRLGSQDMRQALLGCLLFVLSQQHPPDAGEASREALSERRGRLAPQK

SEQ ID NO: 21 - protein, 84 aa, Trichechus manatus
MPAPLLSLLLRALLSRLLLPAARLARQHLLPLLRRLARRLGSQDIREALLGCLLFVLSQRHPPDAGEASRVALSERRERLDPQK

SEQ ID NO: 22 - protein, 84 aa, Unknown
  Tenrecidae
MPAPLLSVLLRALLSRLLLPAARLARQHLLPLLRRLARRLGSQDVREALLGCLLFVLSQRHPPDAGEASAVARSEKRERLAPRK

SEQ ID NO: 23 - protein, 84 aa, Bradypus variegatus
MPAPLLPLLLRSLLSRLLLPAARLARQHLLPLLRRLARRLAAQDMREALLGCLLFVLSQRHPPDAGEPSRVAHPEKRERLAPSK

SEQ ID NO: 24 - protein, 79 aa, Unknown
  Dasypodidae
MPVPLLQPLLRALLSLLLLPAARLARQRLLPLLRRLAGHLISQNTREALLGCLLAILNHRQPEDAGEASGVPRPERREG

SEQ ID NO: 25 - protein, 75 aa, Didelphis virginiana
MPVPLLPLLLRTLLSRLLLPTARLARKHLVPLLRRLARRLGSQETRQALLGCLIYALGLRHQPDAERNGVESKGG

SEQ ID NO: 26 - protein, 62 aa, Unknown
  Testudines
MPAPLLLLPLLRTLLARLLLLARKRLLPLLRRLGARLGSRESREAVLTCLLCILNLRKKADD

SEQ ID NO: 27 - protein, 62 aa, Gallus gallus
MPAVPLAAPLLRALLARLLQLARKRLLPPLRRLGRRLRSRESRQALLTCLLCILNLHKKADA

SEQ ID NO: 28 - protein, 80 aa, Xenopus tropicalis
MPVPFLLLRTLLVRAAGSRLAVSGARQLTKGARWARSHLLVLLQRLWARITSEETRQALLGCVLCLLNLQHKSNTDTGAH

SEQ ID NO: 29 - protein, 75 aa, Danio
MPAVFLLLRSLVVRLFGSRLAASGVQLLRRILTTATGHLGTVLRNIWERISSQQSKEAILGCVLCLLNMHKKVDN

SEQ ID NO: 30 - protein, 75 aa, Unknown
  Coelacanthiformes
MPALFLLLRTLFIRLLGSQLAVSAGLFLRRSLAAAGGRLAALLRRVWDRIRSEESRQVALSCVLCILNLHKKVDE

SEQ ID NO: 31 - protein, 75 aa, Unknown
  Chimaeriformes
MCALLCLRALLLRILRSKLTASALEFIRRRSLAAGSRLGSLLRAGWSRLTRERSRAAVLSCVLCLLDMHKRDTDK

SEQ ID NO: 32 - DNA, 20 bp, Artificial Sequence
  Synthetic primer
gctgctgcctgttgcccgcc

SEQ ID NO: 33 - DNA, 19 bp, Artificial Sequence
  Synthetic primer
aggcctctccagaatccgg

SEQ ID NO: 34 - DNA, 21 bp, Artificial Sequence
  Synthetic primer
agttcaggcttcaggtcagag

SEQ ID NO: 35 - DNA, 20 bp, Artificial Sequence
  Synthetic primer
gctaggggagtgggaactgt

SEQ ID NO: 36 - DNA, 21 bp, Artificial Sequence
  Synthetic primer
gttagaactggtgagcaggag

SEQ ID NO: 37 - DNA, 18 bp, Artificial Sequence
  Synthetic primer
ccatcgggagcaatggaa

SEQ ID NO: 38 - DNA, 18 bp, Artificial Sequence
  Synthetic primer
cctgctgtctctcccaag

SEQ ID NO: 39 - DNA, 19 bp, Artificial Sequence
  Synthetic primer
agaaccagtgggtccctaa

SEQ ID NO: 40 - DNA, 21 bp, Artificial Sequence
  Synthetic primer
ccactccgggacatagacttg

SEQ ID NO: 41 - DNA, 20 bp, Artificial Sequence
  Synthetic primer
aaaagcgcaggtctggtgag

SEQ ID NO: 42 - DNA, 23 bp, Artificial Sequence
  Synthetic primer
gagacatccccctatttctacca

SEQ ID NO: 43 - DNA, 19 bp, Artificial Sequence
  Synthetic primer
gctcagtccgctcatagcc

SEQ ID NO: 44 - DNA, 21 bp, Artificial Sequence
  Synthetic primer
ggagaggattgaggcccaaaa

SEQ ID NO: 45 - DNA, 21 bp, Artificial Sequence
  Synthetic primer
cacggtcactttccctccatc

SEQ ID NO: 46 - DNA, 21 bp, Artificial Sequence
  Synthetic primer
accgcagctaggaataatgga

SEQ ID NO: 47 - DNA, 19 bp, Artificial Sequence
  Synthetic primer
gcctcagttccgaaaacca

SEQ ID NO: 48 - DNA, 21 bp, Artificial Sequence
  Synthetic primer
agttcaggcttcaggtcagag

SEQ ID NO: 49 - DNA, 20 bp, Artificial Sequence
  Synthetic primer
gctaggggagtgggaactgt

SEQ ID NO: 50 - DNA, 20 bp, Artificial sequence
  Synthetic oligonucleotide
ctgcctgttgccgcctggcc

SEQ ID NO: 51 - DNA, 19 bp, Artificial sequence
  Synthetic oligonucleotide
ctgcctgttgcctggcccg

SEQ ID NO: 52 - DNA, 20 bp, Artificial sequence
  Synthetic oligonucleotide
ctgcctgttgccgcctggcc

SEQ ID NO: 53 - DNA, 20 bp, Artificial sequence
  Synthetic oligonucleotide
gccagcaacagattctggag

SEQ ID NO: 54 - DNA, 20 bp, Artificial sequence
  Synthetic oligonucleotide
ctgttgcccgattctggaga

SEQ ID NO: 55 - DNA, 22 bp, Artificial sequence
  Synthetic oligonucleotide
ggttctggaagaggaccactgg

SEQ ID NO: 56 - DNA, 20 bp, Artificial sequence
  Synthetic oligonucleotide
aacagccaccagtgggcgaa

SEQ ID NO: 57 - DNA, 20 bp, Artificial sequence
  Synthetic oligonucleotide
ctgctgcctggaggaaggag

(19) United States
(12) Patent Application Publication
(10) Pub. No.: US 2019/0382732 A1
(43) Pub. Date: Dec. 19, 2019

(54) COMPOSITIONS AND METHODS RELATING TO MYOMIXER-PROMOTED MUSCLE CELL FUSION

(71) Applicant: The Board of Regents of the University of Texas System, Austin, TX (US)

(72) Inventors: Eric N. OLSON, University Park, TX (US); Pengpeng BI, Dallas, TX (US)

(73) Assignee: The Board of Regents of the University of Texas System, Austin, TX (US)

(21) Appl. No.: 16/487,937

(22) PCT Filed: Feb. 14, 2018
(86) PCT No.: PCT/US2018/018137

§ 371 Date: Aug. 22, 2019

Related U.S. Application Data
(60) Provisional application No. 62/463,365, filed on Feb. 24, 2017.

(51) Int. Cl. C12N 5/16; C12N 5/077; C12N 15/11; C12N 15/85; C12N 9/22; A61K 35/34; C07K 14/47

(52) U.S. Cl. CPC C12N 5/16; C12N 5/0656; C12N 15/11; C12N 15/85; C12N 9/22; A61K 35/34; C07K 2319/43; C12N 2310/20; C12N 2800/80; C12N 2320/32; C12N 2502/1335; C12N 2510/00; C12N 2740/16043; C07K 14/4716

(57) ABSTRACT

The present disclosure describes the fusogenic promoting activity of the Myomixer protein. This polypeptide, when expressed in non-muscle cells with the Myomaker protein, is able to increase fusion of the cell with a muscle cell, but not with other non-muscle cells, as compared to cells only expression the Myomaker protein. The use of this protein and cells expressing it in the delivery of exogenous genetic material to muscle cells also is described.

### PRIORITY CLAIM

This application claims benefit of priority to U.S. Provisional Application Ser. No. 62/463,365, filed Feb. 24, 2017, the entire contents of which are hereby incorporated by reference.

### GOVERNMENT FUNDING CLAUSE

This invention was made with government support under grant no. R01AR067294-01 awarded by the National Institutes of Health. The government has certain rights in the invention.

### BACKGROUND

### 1. Field

The present disclosure relates generally to the fields of developmental biology, cell biology and molecular biology. More particularly, it relates the muscle cell fusion promoting activity of the Myomixer protein.

### 2. Description of Related Art

Skeletal muscle is the largest tissue in the body, accounting for ˜40% of human body mass. The formation of skeletal muscle involves a series of events that begins with the specification of muscle cell fate by Pax7 and MyoD, followed by the expression of a vast number of genes that establish muscle structure and function (Bentzinger et al., 2012; Buckingham et al., 2014). A fundamental step in this process is the fusion of mono-nucleated myoblasts to form multi-nucleated myofibers (Rochlin et al., 2010; Demonbreun et al., 2015; Krauss et al., 2017; Simionescu et al., 2011; Abmayr & Pavlath, 2012). Similarly, in response to injury, myogenic progenitor cells within the adult musculature are activated and fuse and generate new myofibers (Yin et al., 2013; Doles & Olwin, 2015; Brack & Rnado, 2012). Whereas many of the initial steps of myoblast fusion are similar to those of other fusogenic cell types (Chen & Olson, 2005), the components and molecular basis of fusion of specific cell types, such as myoblasts, have not been fully defined.

Myoblast fusion is a complex and tightly controlled process required for the formation of skeletal muscle fibers (Chen and Olson, 2005). The fusion process must be highly cell type-specific to ensure that fusogenic myoblasts do not form syncytia with non-muscle cell types. While the transcriptional mechanisms governing skeletal muscle development have been elucidated in detail (Bentzinger et al., 2012; Berkes and Tapscott, 2005; Buckingham 2006; Kang and Krauss, 2010), the mechanisms that coordinate myoblast fusion remain poorly understood, and no muscle-specific protein that directly regulates myoblast fusion has been identified in any organism (Abmayr & Pavlath, 2012; Rochlin et al., 2010). In contrast, numerous proteins involved in cell-cell adhesion and actin dynamics have been implicated in myoblast fusion (Charrasse et al., 2007; Charrasse et al., 2002; Schwander et al., 2003; Griffinet et al., 2009; Yagami-Hiromasa et al., 1995). However, none of these proteins are muscle-specific, necessary and sufficient for mammalian myoblast fusion, suggesting that muscle-specific components of this process remain to be discovered.

### SUMMARY

Thus, in accordance with the present disclosure, there is provided a cell transformed with an exogenous nucleic acid encoding a Myomixer polypeptide under the control of a promoter active in said cell. The cell may be a human cell, a non-muscle cell, a fibroblast, bone marrow cell or blood cell. The exogenous nucleic acid may be under the control of constitutive promoter or an inducible promoter. The cell may e further transformed with exogenous nucleic acid encoding a Myomaker polypeptide is under the control of a promoter active in said cell. The exogenous nucleic acid may be incorporated into a chromosome of said cell. The exogenous nucleic acid may be carried episomally by said cell. The cell may express a detectable marker and/or a selectable marker. The cell may be transformed to express a gene of interest other than Myomixer, such as a therapeutic gene or a marker gene.

In another embodiment, there is provided a method of preparing a non-muscle cell fusion partner comprising transferring into a non-muscle cell a nucleic acid encoding an exogenous Myomixer protein under the control of a promoter active in said cell. The cell may be stably transformed, or transiently transfected. The method may further comprise transferring into said cell a nucleic acid encoding or sufficient to produce a detectable marker. The exogenous nucleic acid may be under the control of constitutive promoter or an inducible promoter. The cell may be further transformed with exogenous nucleic acid encoding a Myomaker polypeptide is under the control of a promoter active in said cell. The cell may be a human cell, a fibroblast, bone marrow cell or blood cell. The exogenous nucleic acid may further encode a selectable marker. The cell may be transformed to express a gene of interest other than Myomixer, such as a therapeutic gene or a marker gene.

In yet another embodiment, there is provided a method of fusing a non-muscle cell to a muscle cell comprising (a) providing a non-muscle cell expressing (i) an exogenous Myomixer protein in said non-muscle cell and (ii) a Myomaker protein; and (b) contacting said non-muscle cell with a muscle, wherein said non-muscle cell expressing Myomixer protein will fuse with said muscle cell. The non-muscle cell may be a human cell, a fibroblast, bone marrow cell or blood cell. Step (b) may be performed in vitro or performed in vivo. The non-muscle cell may express a detectable marker or selectable marker. The non-muscle cell may be further transformed with exogenous nucleic acid encoding a Myomaker polypeptide is under the control of a promoter active in said cell. The muscle cell may be an isolated muscle cell, may be a muscle cell is located in intact muscle tissue, and/or may be a myoblast.

In a further embodiment, there is provided a method of delivering a gene of interest to a muscle cell comprising (a) providing a non-muscle cell expressing an exogenous Myomixer protein and a Myomaker protein, wherein said non-muscle cell further comprises a gene of interest; and (b) contacting said non-muscle cell with a muscle, wherein said non-muscle cell expressing Myomixer and Myomaker proteins will fuse with said muscle cell and deliver said gene of interest to said muscle cell. The non-muscle cell may be a human cell, a fibroblast, bone marrow cell or blood cell. Step (b) may be performed in vitro or performed in vivo.

The non-muscle cell may express a detectable marker or a selectable marker. The non-muscle cell may be further transformed with exogenous nucleic acid encoding a Myomaker polypeptide is under the control of a promoter active in said cell. The muscle cell may be an isolated muscle cell, or may be located in intact muscle tissue, or may be a myoblast. The muscle cell may exhibit a pathologic phenotype, and said gene of interest correct said genotype, such as wherein said pathologic phenotype is the underexpression or absence of a normal gene product, or the expression of a defective gene product.

The muscle cell may have a pathologic phenotype associated with congenital myopathy, sarcopenia, amyotrophic lateral sclerosis, muscular dystrophy, Pompe disease or rhabdomyosarcoma. The non-muscle cell may be delivered to an affected muscle tissue comprising said muscle in a subject, such as via intramuscular injection, and/or wherein delivery is repeated at least once, and/or wherein a secondary therapy is administered to said subject. The non-muscle cell may be delivered to said muscle cell ex vivo and subsequently implanted in to a subject, such as where muscle cell is comprised in intact muscle tissue.

In still a further embodiment, there is provided an expression cassette comprising an exogenous nucleic acid encoding a Myomixer polypeptide under the control of a promoter active in eukaryotic cells. The exogenous nucleic acid may be under the control of a constitutive promoter or an inducible promoter. The expression cassette may further comprise an exogenous nucleic acid encoding a Myomaker polypeptide under the control of a promoter active in eukaryotic cells. The expresses may further encode a detectable marker and/or a selectable marker. The expression cassette may comprise an exogenous nuclei acid encoding a gene of interest other than Myomixer or Myomaker. The expression cassette may comprised in a replicable vector, such as a viral vector (e.g., a retroviral vector, a lentiviral vector, an adenoviral vector or an adeno-associated viral vector), or a non-viral vector (e.g., non-viral vector formulated in a liposome or nanoparticle).

In an additional embodiment, there it provided a method of correcting a genetic defect in a cell in a subject comprising (a) providing a non-muscle cell from said subject; (b) introducing one or more expression cassettes into said non-muscle cell expressing (i) exogenous Myomixer and Myomaker proteins in said non-muscle cell and (ii) one or more therapeutic genes; and (c) contacting said non-muscle cell with a muscle cell having a genetic defect, wherein said non-muscle cell expressing Myomixer and Myomaker proteins will fuse with said muscle cell and deliver said therapeutic gene or genes to said muscle cell, thereby correcting said genetic defect. The non-muscle cell may be a human cell, a fibroblast, bone marrow cell or blood cell. Steps (b) and/or (c) may be performed in vitro or in vivo, or step (b) may be performed in vitro and step (c) may be performed in vivo.

The non-muscle cell may express a detectable marker or a selectable marker. The therapeutic gene or genes may comprise Cas9 and at least one therapeutic sgRNA. The muscle cell may be an isolated muscle cell. The muscle cell may be is located in intact muscle tissue. The genetic defect may be a Duchenne Muscular Dystrophy mutation, a congenital myopathy, Pompe disease, or amyotrophic lateral sclerosis. The non-muscle cell may be delivered to an affected muscle tissue comprising said muscle in a subject, such as by intramuscular injection. Delivery may be repeated at least once. A secondary therapy may be administered to said subject.

The non-muscle cell maybe contacted with said muscle cell ex vivo and subsequently implanted in to a subject. The muscle cell may be comprised in intact muscle tissue. The muscle cell may be a myoblast. The expression cassette or cassettes may comprise a constitutive promoter or an inducible promoter. The expression cassette or cassettes may encode a detectable marker and/or a selectable marker. The expression cassette may be comprised in a replicable vector, such as a viral vector (e.g., a retroviral vector, a lentiviral vector, an adenoviral vector or an adeno-associated viral vector) or a non-viral vector, such as a non-viral vector formulated in a liposome or nanoparticle.

As used herein the specification, “a” or “an” may mean one or more. As used herein in the claim(s), when used in conjunction with the word “comprising,” the words “a” or “an” may mean one or more than one. As used herein “another” may mean at least a second or more. Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-F. Myomixer is a membrane protein and essential for myoblast fusion. (FIG. 1A) WT and Myomixer KO primary myoblasts were differentiated for one week, and stained with MY32 antibody (myosin) and Hoechst showing requirement of Myomixer for myoblast fusion. Scale bar, 50 μm. (FIG. 1B) Quantification of nuclei in WT and Myomixer KO primary myoblast cultures (n=3) (graph key is top to bottom/left to right). (FIG. 1C) Western blot showing Myomixer, Myosin and Gapdh expression in WT or Myomixer KO primary myoblast cultures after 4 days in DM. (D) Amino acid sequence of Myomixer and cross-species homology (SEQ ID NOs: 9-31). Basic residues are blue; acidic residues arered and leucines are yellow. Conserved amino acids are highlighted. ss, secondary structure; HH, helix; CC, coil. (FIG. 1E) Live cell staining of Myomixer transfected C2C12 myoblasts showing cell surface localization of Myomixer with a C-terminal FLAG tag. Laminin was stained after FLAG staining and permeabilization. (FIG. 1F) Western blot analysis of cytosolic (C) and membrane (M) fractions of retrovirus-Myomixer infected 293 cells and WT C2C12 cells (day 4 post-differentiation). α-Tubulin and Gapdh blots were used as positive controls of cytosolic proteins. Insulin receptor β, N-Cadherin, EGF receptor blots were used as positive controls of membrane proteins. *P<0.05, ***P<0.001, Student's t test.

FIGS. 2A-H. Myomixer is specifically expressed during muscle development and adult muscle regeneration. (FIG. 2A) Western blot showing Myomixer, Myosin and Gapdh expression during C2C12 differentiation. (FIG. 2B) RNA-seq data of Myomixer in Pax7+ and Twist2+ skeletal muscle progenitors in growth medium (GM) and 2 days after switching to differentiation medium (DM) (graph key is top to bottom/left to right). FPKM: fragments per kilobase of transcript per million mapped reads. (FIG. 2C) Expression of Myomixer transcripts during tongue muscle development detected by qPCR (n=4). (FIG. 2D) Western blot showing Myomixer and Gapdh expression in the indicated tissues. SKM, skeletal muscle. (FIG. 2E) In situ hybridization showing Myomixer transcript expression in transverse sections of mouse embryos at E12.5 and E15.5. Scale bar, 250 μm (a), 500 μm (b), 200 μm (c). Image c is a magnification of the boxed area shown in image b. M: Myotome derived muscle mass; Sc, scapular premuscle masses; EP, extensor muscle primordium; FP, flexor muscle primordium; IP, intercostal muscle primordium; I, intercostal muscles; TB, triceps brachii; P, pectoralis profundus; ECRL, extensor carpi radialis longus; B, brachioradialis; R, radius; PL, palmaris longus. (FIG. 2F) Myomixer mRNA expression in CTX-injured skeletal muscle from 1-month old WT mice, as determined by qPCR. (n=3). (FIG. 2G) Cross-section of CTX-injured muscles at day 4 and day 7 showing Myomixer (red) and myosin (MY32: green) immunostaining. Scale bar, 20 μm. (FIG. 2H) Up-regulation of Myomixer mRNA expression in mdx compared to WT muscle, as detected by qPCR (n=4). *P<0.05, ***P<0.001, Student's t test.

FIGS. 3A-E. Myomixer is essential for muscle development. (FIG. 3A) WT and Myomixer KO embryos at E17.5 dissected and skinned to reveal the lack of muscle in Myomixer KO limbs. (FIG. 3B) H&E staining of E17.5 limb muscles shows lack of muscle fibers in Myomixer KO embryos. Scale bars, 50 μm. (FIG. 3C) Immunohistochemistry of E17.5 limb muscles using MY32 (myosin) antibody. WT myofibers show multi-nucleation which is absent in Myomixer KO sections. Scale bars, 50 μm. (FIG. 3D) Genotyping shows different types of deletions in the Myomixer sgRNA targeting region. WT band is 743 bp. (FIG. 3E) Western blot analysis of Myomixer and Gapdh in forelimb tissues of E17.5 WT and Myomixer KO embryos.

FIGS. 4A-H. Myomixer binds Myomaker and synergizes to induce cell fusion. (FIG. 4A) Co-immunoprecipitation assays were performed using 10T1/2 cells or C2C12 myoblasts infected with retroviruses expressing Myomixer and/or FLAG-Myomaker. IP, immunoprecipitation; IB, immunoblot. (FIG. 4B) MY32 (myosin) immunostaining of C2C12 cells infected with retroviruses expressing Myomaker, Myomixer or both, and differentiated for 4 days. Nuclei were counterstained with Hoechst. Scale bar, 50 μm. (FIG. 4C) Quantification of nuclei in C2C12 cells in B (n=3) (graph key is top to bottom/left to right). (FIG. 4D and FIG. 4E) GFP labelled 10T1/2 fibroblasts infected with retroviruses expressing Myomaker, Myomixer or both. Two days after infection, cells were mixed with Cherry-labeled C2C12 cells and differentiated for one week. Fluorescence images of GFP, RFP (Cherry) and Hoechst to counterstain nucleus. Scale bar, 100 μm. (FIG. 4F) Overexpression of Myomixer and Myomaker in 10T1/2 fibroblasts detected by western blots. (FIG. 4G) Summary of fusogenic activities of Myomixer and Myomaker in different cell types. (FIG. 4H) Fluorescence images of GFP and MY32 (myosin) immunostaining (red) of mixed cultures of C2C12 cells (WT or Myomixer KO) with 10T1/2-GFP fibroblasts infected with retroviruses expressing Myomaker and/or Myomixer, following one week of differentiation. Nuclei were counterstained with Hoechst. Scale bar, 50 μm.

FIGS. 5A-J. CRISPR screening strategy and validation of Myomixer single guide (sg) RNAs. (FIG. 5A) Schematic of the genome-wide CRISPR loss-of-function screen. C2C12 myoblasts were infected with recombinant lentiviruses expressing Cas9 and sgRNAs. Addition of puromycin selects for cells that have genome integration of viral DNA, thereafter stably expressing Cas9 and sgRNA to knockout its target gene. Cells were switched to differentiation media (low serum) for one week. Trypsin digestion was used to separate myotubes from myoblasts. Sequencing identified the screen “hits”. If the gene is essential for differentiation or fusion of C2C12 myoblasts, loss-of-function of this gene will block myotube formation and these altered cells will appear in the myoblast population. The ratio of the sgRNAs identified in myoblasts versus myotubes reflects the function of its target gene in myoblast differentiation or fusion. (FIG. 5B) Western blot analysis using MY32 antibody shows enrichment of myosin heavy chain expression in trypsin separated myotubes (low trypsin) compared to myoblasts (high trypsin). (FIG. 5C) List of selected genes (“hits”) identified in the screen. Hits (enrichment score >2.5) were compared to genes upregulated (>2.5-fold) during differentiation of Pax7+ satellite cells and Twist2+ myogenic progenitors, as well as, genes up-regulated (>1.5-fold) during C2C12 differentiation (GSE4694). (FIG. 5D) Analysis of chromatin immunoprecipitation sequencing data (GSM915183, GSM915185, GSM915186, GSM915165, GSM915159, GSM915163, GSM915166, GSM915164) to show occupancy dynamics of Myod and Myogenin on the Myomixer promoter during C2C12 differentiation. (FIG. 5E) The Myomixer gene spans three exons with the ORF confined to exon 3. Positions of sgRNAs and primers for genotype analysis are shown. (FIG. 5F) Sequencing of genomic PCR fragments from C2C12 cells following disruption of Myomixer by CRISPR (SEQ ID NOS: 50-54). (FIG. 5G) Immunostaining of WT and Myomixer KO C2C12 cells with MY32 (myosin heavy chain) antibody and Hoechst stain shows requirement of Myomixer for fusion. Scale bar, 50 μm. (FIG. 5H) Quantification of nuclei in WT and Myomixer KO C2C12 cells (n=3) (graph key is top to bottom/left to right). (FIG. 5I) Western blot showing Myomixer, Myosin and Gapdh expression in WT and Myomixer KO C2C12 cells. (FIG. 5J) RNA expression of the indicated transcripts in WT and Myomixer KO C2C12 cells detected by qPCR (graph key is top to bottom/left to right). *P<0.05, ***P<0.001, Student's t test.

FIG. 6. Gene expression during C2C12 cells differentiation. RNA was isolated from C2C12 cells differentiated at the indicated time and Myogenin or Myomixer expression was measured by qPCR (n=3) (graph key is top to bottom/left to right).

FIG. 7. Sequence of genomic DNA from Myomixer KO embryos. Myomixer KO embryos at E17.5 were isolated and genomic DNA sequence was determined from tail DNA. (SEQ ID NOS: 55-57)

FIGS. 8A-B. Measuring Myomaker expression. Using qPCR, Myomaker expression was measured in (FIG. 8A) WT and Myomixer KO C2C12 cells (n=3) and (FIG. 8B) WT and Myomixer KO E17.5 mouse hindlimb (n=6 for WT and 9 for KO). For both panels, graph key is top to bottom/left to right.

FIG. 9. Myomaker Protein and DNA Sequences.

FIG. 10. Myomixer Protein and DNA Sequences.

### DESCRIPTION OF ILLUSTRATIVE EMBODIMENTS

There are multiple types of membrane fusion, including virus-cell fusion, intracellular vesicle fusion, and cell-cell fusion (Chen and Olson 2005). Similarities exist between different fusion mechanisms, but relatively little is known about cell-cell fusion compared to other fusion processes, especially with respect to the fusogenic proteins that directly merge intercellular membranes.

Skeletal muscle formation occurs through fusion of myoblasts to form multi-nucleated myofibers. From a genome-wide CRISPR loss-of-function screen for genes required for myoblast fusion and myogenesis, the inventors have discovered a small muscle-specific peptide that they call Myomixer. Myomixer expression coincides with myoblast differentiation in vitro and in vivo and is essential for fusion and skeletal muscle formation during embryogenesis. Myomixer localizes to the plasma membrane where it promotes cell-cell fusion through its association with Myomaker, a fusogenic membrane protein. The inventors conclude that the Myomixer-Myomaker pair controls the critical step in myofiber formation during muscle development.

The discovery of Myomixer builds on the inventors' previous discovery of Myomaker as a potent myoblast fusion protein. The ability to enhance Myomaker activity further facilitates the exploitation of this underlying fundamental cellular processes, and enhance the ability of Myomaker to drive fusion of non-muscle cells with muscle cells represents an interesting strategy for enhancing muscle repair. These and other aspects of the disclosure are described below.

### I. MYOMAKERAND MYOMIXER

A. Myomaker

Transmembrane protein 8c (Tmem8c), designated Myomaker, is a poorly characterized protein of 221 residues that highly conserved across vertebrates. The gene is located on human chromosome 9q34.2. It contains 6 putative helical regions of roughly 20 amino acids distributed evenly throughout the protein. The DNA and protein sequences for mouse Myomaker are provided as SEQ ID NO: 1 and 2, respectively, and the DNA and protein sequences for humanMyomaker are provided as SEQ ID NO: 3 and 4, respectively.

Myomaker is transiently expressed during myoblast fusion and is both necessary and sufficient to drive merger of plasma membranes in vivo and in vitro. Myomaker is a muscle-specific plasma membrane protein expressed specifically during times of myoblast fusion, and required for the formation of multinucleated myofibers. While surface glycoproteins, including cadherins, β-1 integrin, MOR23, and Adam12 (Charrasse et al., 2007, Charrasse et al., 2002, Schwander et al., 2003, Griffinet et al., 2009 and Yagami-Hiromasa et al., 1995), have been shown to influence myoblast fusion, Myomaker is the only muscle-specific protein yet identified that is absolutely essential for myoblast fusion in vivo. The absence of multinucleated myofibers in Myomaker^{-/-} mice demonstrates the requirement of this membrane protein for the formation of all skeletal muscles.

Myoblast fusion is a multistep process requiring intimate cell-cell interaction followed by membrane coalescence accompanied by actin-cytoskeletal dynamics that drive cell merger. Myomaker clearly participates in the membrane fusion reaction, as demonstrated by its ability to stimulate myoblast fusion and the fusion of fibroblasts to myoblasts. The inability of Myomaker alone to induce fusion of fibroblasts suggests it may require activation or additional myoblast proteins to exert its fusogenic activity, likely reflecting a requirement for close membrane apposition to allow membrane merger. Further evidence that additional myoblast proteins are required for fusion is the inventors' finding that WT myoblasts can fuse with Myomaker^{-/-} myoblasts. The requirement for interactions between membrane proteins on opposite cells during myoblast fusion has been shown in zebrafish and Drosophila (Abmayr and Pavlath 2012 and Powell and Wright 2011), suggesting the molecular regulation of myoblast fusion differs from that of virus-cell fusion, which mainly requires the expression of a fusogenic protein (Oren-Suissa & Podbilewicz). Changes in the actin-cytoskeleton are required for cell-cell fusion (Wilson and Snell 1998; Shilagardi et al., 2013). Consistent with this paradigm, the activity of Myomaker is abolished by cytochalasin D and latrunculin B, which disrupt cytoskeletal events required for fusion, indicating that Myomaker depends on the cytoskeleton to exert its function.

B. Myomixer

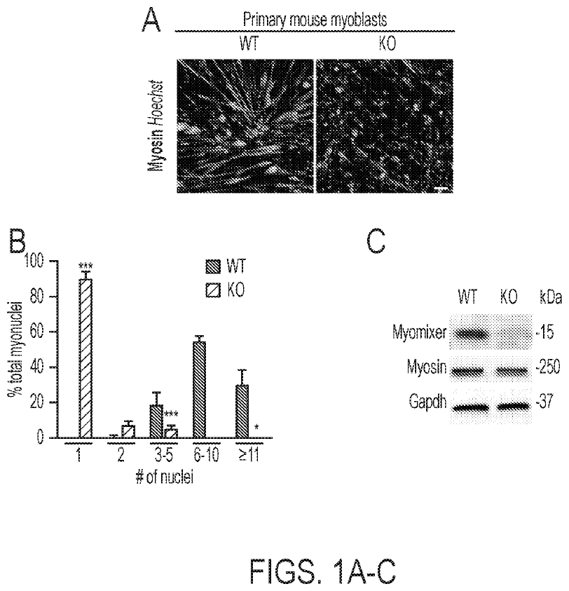

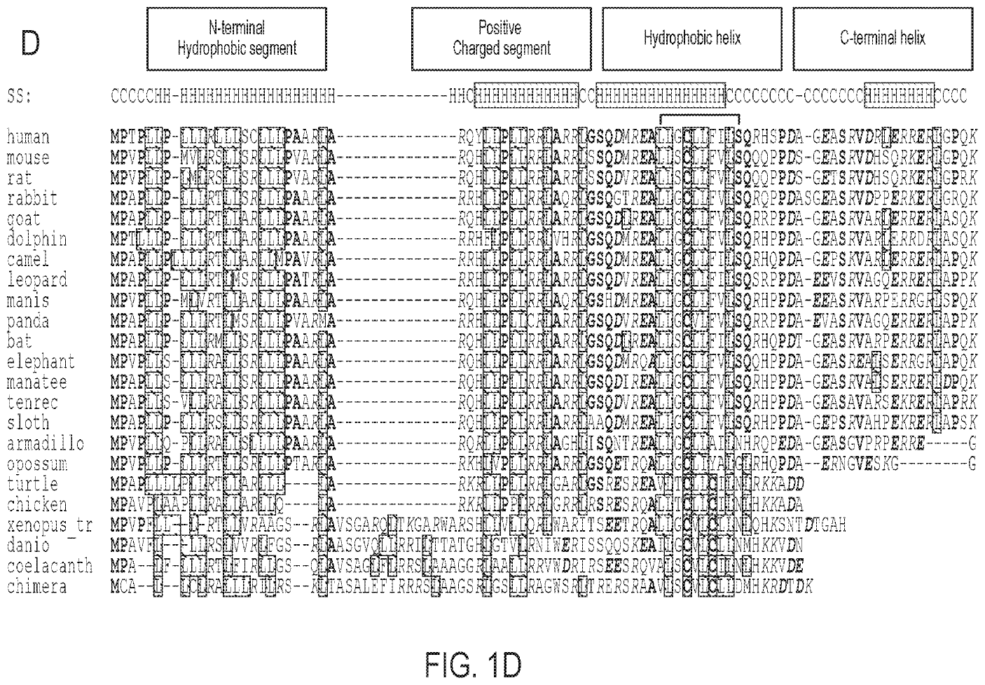

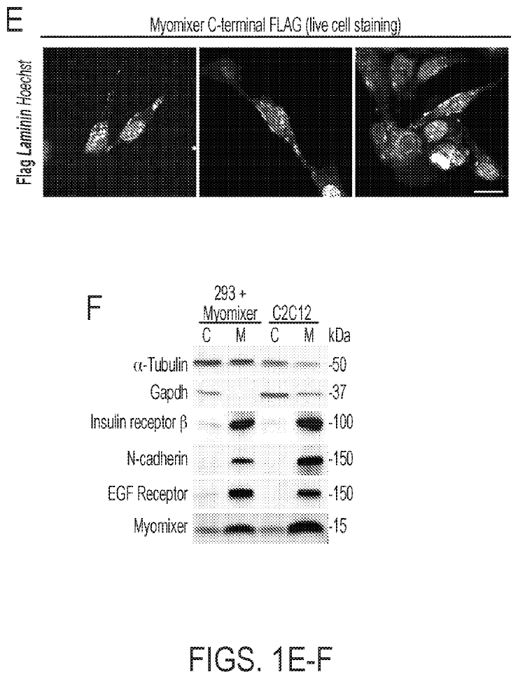

Mouse Myomixer is a micropeptide that is 84 amino acids long (FIG. 1D). Orthologs of Myomixer are conserved across diverse vertebrate species (FIG. 1D). The small size of Myomixer places it in the category of micropeptides, characterized by unprocessed ORFs of less than 100 amino acids (Marquez-Medina et al., 2015). It is noteworthy, in this regard, that a majority of micropeptides identified to date are embedded in membranes (Anderson et al., 2015; 2016). Protein and DNA sequences for mouse Myomixer are set forth in SEQ ID NOS: 5 and 6, respectively. Protein and DNA sequences for human Myomixer are set forth in SEQ ID NOS: 7 and 8, respectively.

Myomixer amino acid sequence is not annotated in fish or amphibians, presumably because ORFs smaller than 100 amino acids are not typically annotated. However, the inventors identified putative Myomixer orthologs in fish, frog and turtle genomes with conservation of several residues, especially many arginines and amphipathic residues (FIG. 1D). The proteins from the various species contain an N-terminal hydrophobic segment, followed by a positively charged helix and an adjacent hydrophobic helix. Myomixer proteins from mammals and marsupials also contain a unique C-terminal helix that is missing from other organisms (FIG. 1D).

The inventors have not found Myomixer-related genes in Drosophila, which also lacks an obvious Myomaker ortholog, suggesting that the Myomixer-Myomaker protein partnership is an invention of higher vertebrates. Perhaps the formation of the large muscles of vertebrates requires this robust fusogenic machinery superimposed upon the basic cell-cell fusion events of simpler organisms such as Drosophila.

The inventors note some striking similarities between Myomixer and the heart-specific micropeptide, DWORF (Dwarf open reading frame), which localizes to the sarcoplasmic reticulum of cardiomyocytes where it associates with the SERCA calcium ATPase and stimulates its activity (Nelson et al., 2016). The inventors speculate that the activities of many membrane proteins may be governed by association with micropeptides that are as yet unidentified.

Because the N-terminus contains an extended hydrophobic segment, thus may represent a putative a membrane anchor. Immunostaining of intact C2C12 myoblasts expressing Myomixer with FLAG tag at the C-terminus revealed the presence of the protein on the cell surface (FIG. 1E). Consistent with these findings, fractionation of C2C12 myotubes into membrane and cytoplasmic fractions showed the localization of Myomixer to membranes (FIG. 1F). Similarly, in 293 cells infected with a Myomixer-expressing retrovirus, the protein was preferentially localized to the membrane (FIG. 1F).

### II. PEPTIDES AND POLYPEPTIDES

In certain embodiments, the present disclosure may concerns Myomixer protein molecules. As used herein, a “protein” or “polypeptide” generally refers full length proteins. In contrast, a peptide is defined as being usually from about 3 to about 100 amino acids. All the “proteinaceous” terms described above may be used interchangeably herein. A human Myomixer polypeptide sequence is provided in SEQ ID NO: 7, while mouse Myomixer polypeptide sequence is provided in SEQ ID NO: 5.

In certain embodiments, the proteinaceous composition comprises at least one protein, polypeptide or peptide. In further embodiments, the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (world-wide-webe at ncbi.nlm.nih.gov). The coding regions for these known genes may be amplified and/or expressed using the techniques disclosed herein or as would be know to those of ordinary skill in the art. Alternatively, proteins may be produced recombinantly or purified from natural sources. Shorter peptide molecules may be synthesized in solution or on a solid support in accordance with conventional techniques. Various automatic synthesizers are commercially available and can be used in accordance with known protocols. See, e.g., Stewart and Young (1984); Tam et al. (1983); Merrifield (1986); and Barany and Merrifield (1979), each incorporated herein by reference.

In certain embodiments, a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

### III. NUCLEIC ACIDS

In certain embodiments of the present disclosure, nucleic acids derived from or encoding Myomixer and Myomaker are provided. In certain aspects, the nucleic acids may comprise wild-type or a modified version of these genes. In particular aspects, the nucleic acid encodes for or comprises a transcribed nucleic acid. In other aspects, the nucleic acid comprises a nucleic acid segment of SEQ ID NO: 6 or 8, or a biologically functional equivalent thereof. In particular aspects, the nucleic acid encodes a protein, polypeptide, or peptide.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an “A,” a “G,” an uracil “U” or a “C”). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single-stranded nucleic acid may be denoted by the prefix “ss,” a double-stranded nucleic acid by the prefix “ds,” and a triple-stranded nucleic acid by the prefix “ts.”

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. In the methods of the present disclosure, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

In certain aspect, the present disclosure concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of Myomixer. Thus, a “nucleic acid segment” may comprise any part of a gene sequence, of from about 10 nucleotides to the full length of Myomixer gene. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

The present disclosure also encompasses a nucleic acid that is complementary to a Myomixer-encoding nucleic acid. In particular embodiments the disclosure encompasses a nucleic acid or a nucleic acid segment complementary to the sequence set forth in SEQ ID NO: 6 or 8. A nucleic acid is a “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

5. Expression Constructs

Within certain embodiments, expression constructs will be are employed to express Myomixer. Expression requires that appropriate signals be provided in vectors, which include various regulatory elements, such as enhancers/promoters from both viral and mammalian sources that drive expression of Myomixer in recipient cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for Myomixer in which part or all of the nucleic acid encoding sequence is capable of being transcribed. The term “vector” is used to refer to a carrier nucleic acid molecule into which a nucleic acid sequence can be inserted for introduction into a cell where it can be replicated. A nucleic acid sequence can be “exogenous,” which means that it is foreign to the particular cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (2001) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996).

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the disclosure, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference.)

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### IV. MUSCLE DISEASES

The present disclosure finds particular relevance in analyzing and treating muscle disorders. In particular, muscle disorders in which muscle cells fail to produce enough (or any) of a gene product needed for normal function, or where an aberrant protein is produce, the use of somatic cell fusion to deliver genes or proteins is highly desired.

The following is a general discussion of a few disorders that could be addressed according to the present disclosure.

A. Muscular Dystrophy

Muscular Dystrophy (MD) is a group of muscle diseases that weaken the musculoskeletal system and hamper locomotion. Muscular dystrophies are characterized by progressive skeletal muscle weakness, defects in muscle proteins, and the death of muscle cells and tissue.

In the 1860s, descriptions of boys who grew progressively weaker, lost the ability to walk, and died at an early age became more prominent in medical journals. In the following decade, French neurologist Guillaume Duchenne gave a comprehensive account of thirteen boys with the most common and severe form of the disease, which now carries his name—Duchenne muscular dystrophy.

It soon became evident that the disease had more than one form. The other major forms are Becker, limb-girdle, congenital, facioscapulohumeral, myotonic, oculopharyngeal, distal, and Emery-Dreifuss muscular dystrophy. These diseases predominantly affect males, although females may be carriers of the disease gene. Most types of MD are multi-system disorders with manifestations in body systems including the heart, gastrointestinal system, nervous system, endocrine glands, eyes and brain.

Apart from the nine major types of muscular dystrophy listed above, several MD-like conditions have also been identified. Normal intellectual, behavioral, bowel and sexual function is noticed in individuals with other forms of MD and MD-like conditions. MD-affected individuals with susceptible intellectual impairment are diagnosed through molecular characteristics but not through problems associated with disability. However, a third of patients who are severely affected with DMD may have cognitive impairment, behavioral, vision and speech problems.

These conditions are generally inherited, and the different muscular dystrophies follow various inheritance patterns. However, mutations of the dystrophin gene and nutritional defects (with no genetics history) at the prenatal stage are also possible in about 33% of people affected by DMD. The main cause of the Duchenne and Becker types of muscular dystrophy is the muscle tissue's cytoskeletal impairment to properly create the functional protein dystrophin and dystrophin-associated protein complex.

Dystrophin protein is found in muscle fibre membrane; its helical nature allows it to act like a spring or shock absorber. Dystrophin links actin (cytoskeleton) and dystroglycans of the muscle cell plasma membrane, known as the sarcolemma (extracellular). In addition to mechanical stabilization, dystrophin also regulates calcium levels.

The diagnosis of muscular dystrophy is based on the results of muscle biopsy, increased creatine phosphokinase (CpK3), electromyography, electrocardiography and DNA analysis.

A physical examination and the patient's medical history will help the doctor determine the type of muscular dystrophy. Specific muscle groups are affected by different types of muscular dystrophy.

Often, there is a loss of muscle mass (wasting), which may be hard to see because some types of muscular dystrophy cause a build up of fat and connective tissue that makes the muscle appear larger. This is called pseudohypertrophy.

There is no known cure for muscular dystrophy, although significant headway is being made with antisense oligonucleotides. Physical therapy, occupational therapy, orthotic intervention (e.g., ankle-foot orthosis), speech therapy and orthopedic instruments (e.g., wheelchairs and standing frames) may be helpful. Inactivity (such as bed rest, sitting for long periods) and bodybuilding efforts to increase myofibrillar hypertrophy can worsen the disease.

There is no specific treatment for any of the forms of muscular dystrophy. Physiotherapy, aerobic exercise, low intensity anabolic steroids, prednisone supplements may help to prevent contractures and maintain muscle tone. Orthoses (orthopedic appliances used for support) and corrective orthopedic surgery may be needed to improve the quality of life in some cases. The cardiac problems that occur with Emery-Dreifuss muscular dystrophy and myotonic muscular dystrophy may require a pacemaker. The myotonia (delayed relaxation of a muscle after a strong contraction) occurring in myotonic muscular dystrophy may be treated with medications such as quinine, phenytoin, or mexiletine, but no actual long term treatment has been found.

Occupational therapy assists the individual with MD in engaging in his/her activities of daily living (self-feeding, self-care activities, etc.) and leisure activities at the most independent level possible. This may be achieved with use of adaptive equipment or the use of energy conservation techniques. Occupational therapy may implement changes to a person's environment, both at home or work, to increase the individual's function and accessibility. Occupational therapists also address psychosocial changes and cognitive decline which may accompany MD, as well as provide support and education about the disease to the family and individual.

High dietary intake of lean meat, sea food, pulses, olive oil, antioxidants; such as leafy vegetables and bell peppers, and fruits like blueberry, cherry etc. is advised. Decreased intake of refined food, trans-fats, and caffeinated and alcoholic beverages is also advised; as is a check for any food allergies.

Diagnosis, neurology, GI-nutrition, respiratory care, cardiac care, orthopedics, psychosocial, rehabilitation, and oral care form the integral part of disease management, all through the patient's life span.

The prognosis for people with muscular dystrophy varies according to the type and progression of the disorder. Some cases may be mild and progress very slowly over a normal lifespan, while others produce severe muscle weakness, functional disability, and loss of the ability to walk. Some children with muscular dystrophy die in infancy while others live into adulthood with only moderate disability. The muscles affected vary, but can be around the pelvis, shoulder, face or elsewhere. Muscular dystrophy can affect adults, but the more severe forms tend to occur in early childhood.

B. Amyotrophic Lateral Sclerosis

Amyotrophic lateral sclerosis (ALS), sometimes called Lou Gehrig's Disease, affects as many as 20,000 Americans at any given time, with 5,000 new cases being diagnosed in the United States each year. ALS affects people of all races and ethnic backgrounds. Men are about 1.5 times more likely than women to be diagnosed with the disease. ALS strikes in the prime of life, with people most commonly diagnosed between the ages of 40 and 70. However, it is possible for individuals to be diagnosed at younger and older ages. About 90-95% of ALS cases occur at random, meaning that individuals do not have a family history of the disease and other family members are not at increased risk for contracting the disease. In about 5-10% of ALS cases there is a family history of the disease.

ALS is a progressive neurological disease that attacks neurons that control voluntary muscles. Motor neurons, which are lost in ALS, are specialized nerve cells located in the brain, brainstem, and spinal cord. These neurons serve as connections from the nervous system to the muscles in the body, and their function is necessary for normal muscle movement. ALS causes motor neurons in both the brain and spinal cord to degenerate, and thus lose the ability to initiate and send messages to the muscles in the body. When the muscles become unable to function, they gradually atrophy and twitch. ALS can begin with very subtle symptoms such as weakness in affected muscles. Where this weakness first appears differs for different people, but the weakness and atrophy spread to other parts of the body as the disease progresses.

Initial symptoms may affect only one leg or arm, causing awkwardness and stumbling when walking or running. Subjects also may suffer difficulty lifting objects or with tasks that require manual dexterity. Eventually, the individual will not be able to stand or walk or use hands and arms to perform activities of daily living. In later stages of the disease, when the muscles in the diaphragm and chest wall become too weak, patients require a ventilator to breathe. Most people with ALS die from respiratory failure, usually 3 to 5 years after being diagnosed; however, some people survive 10 or more years after diagnosis.

Perhaps the most tragic irony of ALS is that it does not impair a person's mind, as the disease affects only the motor neurons. Personality, intelligence, memory, and self-awareness are not affected, nor are the senses of sight, smell, touch, hearing, and taste. Yet at the same time, ALS causes dramatic defects in an individual's ability to speak loudly and clearly, and eventually, completely prevents speaking and vocalizing. Early speech-related symptoms include nasal speech quality, difficulty pronouncing words, and difficulty with conversation. As muscles for breathing weaken, it becomes difficult for patients to speak loud enough to be understood and, eventually, extensive muscle atrophy eliminates the ability to speak altogether. Patients also experience difficulty chewing and swallowing, which increase over time to the point that a feeding tube is required.

C. Pompe Disease

Glycogen storage disease type II (also called Pompe disease or acid maltase deficiency) is an autosomal recessive metabolic disorder which damages muscle and nerve cells throughout the body. It is caused by an accumulation of glycogen in the lysosome due to deficiency of the lysosomal acid alpha-glucosidase enzyme. It is the only glycogen storage disease with a defect in lysosomal metabolism, and the first glycogen storage disease to be identified, in 1932 by the Dutch pathologist J. C. Pompe. The build-up of glycogen causes progressive muscle weakness (myopathy) throughout the body and affects various body tissues, particularly in the heart, skeletal muscles, liver and nervous system.

There are exceptions, but levels of alpha-glucosidase determines the type of GSD II an individual may have. More alpha glucosidase present in the individuals' muscles means symptoms occur later in life and progress more slowly. GSD II is broadly divided into two onset forms based on the age symptoms occur. Infantile-onset form is usually diagnosed at 4-8 months; muscles appear normal but are limp and weak preventing them from lifting their head or rolling over. As the disease progresses heart muscles thicken and progressively fail. Without treatment death usually occurs due to heart failure and respiratory weakness. Late/later onset form occurs later than one to two years and progresses more slowly than Infantile-onset form. One of the first symptoms is a progressive decrease in muscle strength starting with the legs and moving to smaller muscles in the trunk and arms, such as the diaphragm and other muscles required for breathing. Respiratory failure is the most common cause of death. Enlargement of the heart muscles and rhythm disturbances are not significant features but do occur in some cases.

The infantile form usually comes to medical attention within the first few months of life. The usual presenting features are cardiomegaly (92%), hypotonia (88%), cardiomyopathy (88%), respiratory distress (78%), muscle weakness (63%), feeding difficulties (57%) and failure to thrive (53%). The main clinical findings include floppy baby appearance, delayed motor milestones and feeding difficulties. Moderate hepatomegaly may be present. Facial features include macroglossia, open mouth, wide open eyes, nasal flaring (due to respiratory distress), and poor facial muscle tone. Cardiopulmonary involvement is manifest by increased respiratory rate, use of accessory muscles for respiration, recurrent chest infections, decreased air entry in the left lower zone (due to cardiomegaly), arrhythmias and evidence of heart failure. Median age at death in untreated cases is 8.7 months and is usually due to cardiorespiratory failure.

This form differs from the infantile principally in the relative lack of cardiac involvement. The onset is more insidious and has a slower progression. Cardiac involvement may occur but is milder than in the infantile form. Skeletal involvement is more prominent with a predilection for the lower limbs. Late onset features include impaired cough, recurrent chest infections, hypotonia, progressive muscle weakness, delayed motor milestones, difficulty swallowing or chewing and reduced vital capacity. Prognosis depends on the age of onset on symptoms with a better prognosis being associated with later onset disease.

Diagnostic procedures include chest X ray, electrocardiogram and echocardiography. Typical findings are those of an enlarged heart with non specific conduction defects. Biochemical investigations include serum creatine kinase (typically increased 10 fold) with lesser elevations of the serum aldolase, aspartate transaminase, alanine transaminase and lactic dehydrogenase. Diagnosis is made by estimating the acid alpha glucosidase activity in either skin biopsy (fibroblasts), muscle biopsy (muscle cells) or in white blood cells. The choice of sample depends on the facilities available at the diagnostic laboratory. In the late onset form, the findings on investigation are similar to those of the infantile form with the caveat that the creatinine kinases may be normal in some cases. The diagnosis is by estimation of the enzyme activity in a suitable sample.

The disease is caused by a mutation in a gene (acid alpha-glucosidase: also known as acid maltase) on long arm of chromosome 17 at 17q25.2-q25.3 (base pair 75,689,876 to 75,708,272). The number of mutations described is currently (in 2010) 289 with 67 being non-pathogenic mutations and 197 pathogenic mutations. The remainder are still being evaluated for their association with disease. The gene spans approximately 20 kb and contains 20 exons with the first exon being noncoding. The coding sequence of the putative catalytic site domain is interrupted in the middle by an intron of 101 bp. The promoter has features characteristic of a ‘housekeeping’ gene. The GC content is high (80%) and distinct TATA and CCAAT motifs are lacking.

Most cases appear to be due to three mutations. A transversion (T→G) mutation is the most common among adults with this disorder. This mutation interrupts a site of RNA splicing. The gene encodes a protein—acid alpha-glucosidase—which is a lysosomal hydrolase. The protein is an enzyme that normally degrades the alpha-1,4 and alpha-1,6 linkages in glycogen, maltose and isomaltose and is required for the degradation of 1-3% of cellular glycogen. The deficiency of this enzyme results in the accumulation of structurally normal glycogen in lysosomes and cytoplasm in affected individuals. Excessive glycogen storage within lysosomes may interrupt normal functioning of other organelles and lead to cellular injury.

Cardiac and respiratory complications are treated symptomatically. Physical and occupational therapy may be beneficial for some patients. Alterations in diet may provide temporary improvement but will not alter the course of the disease. Genetic counseling can provide families with information regarding risk in future pregnancies.

On Apr. 28, 2006 the US Food and Drug Administration approved a Biologic License Application (BLA) for Myozyme (alglucosidase alfa, rhGAA), the first treatment for patients with Pompe disease, developed by a team of Duke University researchers. This was based on enzyme replacement therapy using biologically active recombinant human alglucosidase alfa produced in Chinese Hamster Ovary cells. Myozyme falls under the FDA Orphan Drug designation and was approved under a priority review. The FDA has approved Myozyme for administration by intravenous infusion of the solution. The safety and efficacy of Myozyme were assessed in two separate clinical trials in 39 infantile-onset patients with Pompe disease ranging in age from 1 month to 3.5 years at the time of the first infusion. Myozyme treatment clearly prolongs ventilator-free survival and overall survival. Early diagnosis and early treatment leads to much better outcomes. The treatment is not without side effects which include fever, flushing, skin rash, increased heart rate and even shock; these conditions, however, are usually manageable.

A new treatment option for this disease is called Lumizyme. Lumizyme and Myozyme have the same generic ingredient (Alglucosidase Alfa) and manufacturer (Genzyme Corporation). The difference between these two products is in the manufacturing process. Today, the Myozyme is made using a 160-L bioreactor, while the Lumizyme uses a 4000-L bioreactor. Because of the difference in the manufacturing process, the FDA claims that the two products are biologically different. Moreover, Lumizyme is FDA approved as replacement therapy for late-onset (noninfantile) Pompe disease without evidence of cardiac hypertrophy in patients 8 years and older. Myozyme is FDA approved for replacement therapy for infantile-onset Pompe disease. The prognosis for individuals with Pompe disease varies according to the onset and severity of symptoms. Without treatment the disease is particularly lethal in infants and young children.

Myozyme (alglucosidase alfa), which helps break down glucose, is a recombinant form of the human enzyme acid alpha-glucosidase, and is also currently being used to replace the missing enzyme. In a study which included the largest cohort of patients with Pompe disease treated with enzyme replacement therapy (ERT) to date findings showed that Myozyme treatment clearly prolongs ventilator-free survival and overall survival in patients with infantile-onset Pompe disease as compared to an untreated historical control population. Furthermore, the study demonstrated that initiation of ERT prior to 6 months of age, which could be facilitated by newborn screening, shows great promise to reduce the mortality and disability associated with this devastating disorder. Taiwan and several states in the United States have started the newborn screening and results of such regimen in early diagnosis and early initiation of the therapy have dramatically improved the outcome of the disease; many of these babies have reached the normal motor developmental milestones.

Another factor affecting the treatment response is generation of antibodies against the infused enzyme, which is particularly severe in Pompe infants who have complete deficiency of the acid alpha-glucosidase. Immune tolerance therapy to eliminate these antibodies has improved the treatment outcome.

A Late Onset Treatment Study (LOTS) was published in 2010. The study was undertaken to evaluate the safety and efficacy of aglucosidase alfa in juvenile and adult patients with Pompe disease. LOTS was a randomized, double-blind, placebo-controlled study that enrolled 90 patients at eight primary sites in the United States and Europe. Participants received either aglucosidase alfa or a placebo every other week for 18 months. The average age of study participants was 44 years. The results showed that, at 78 weeks, patients treated with aglucosidase alfa increased their distance walked in six minutes by an average of approximately 25 meters as compared with the placebo group which declined by 3 meters (P=0.03). The placebo group did not show any improvement from baseline. The average baseline distance walked in six minutes in both groups was approximately 325 meters. Percent predicted forced vital capacity in the group of patients treated with aglucosidase alfa increased by 1.2 percent at 78 weeks. In contrast, it declined by approximately 2.2 percent in the placebo group (P=0.006).

D. Rhabdomyosarcoma

A rhabdomyosarcoma, commonly referred to as RMS, is a type of cancer, specifically a sarcoma (cancer of connective tissues), in which the cancer cells are thought to arise from skeletal muscle progenitors. It can also be found attached to muscle tissue, wrapped around intestines, or in any anatomic location. Most occur in areas naturally lacking in skeletal muscle, such as the head, neck, and genitourinary tract.

Its two most common forms are embryonal rhabdomyosarcoma and alveolar rhabdomyosarcoma. In the former, which is more common in younger children, the cancer cells resemble those of a typical 6-to-8-week embryo. In the latter, which is more common in older children and teenagers, they resemble those of a typical 10-to-12-week embryo.

Rhabdomyosarcoma is a relatively rare form of cancer. It is most common in children ages one to five, and is also found in teens aged 15 to 19, although this is more rare. This cancer is also an adult cancer but it is rare. St. Jude Children's Research Hospital reports that rhabdomyosarcoma is the most common soft tissue sarcoma in children. Soft tissue sarcomas make up about 3% of childhood cancers.

The diagnosis of rhabdomyosarcoma is made by a pathologist, he or she will examine a biopsy of the tumor under the microscope and arrive at the diagnosis of rhabdomyosarcoma based on the morphology (appearance) of the tumor cells and the results of immunohistochemical stains. Diagnosis of rhabdomyosarcoma depends on recognition of differentiation toward skeletal muscle cells. The proteins myoD1 and myogenin are transcription factor proteins normally found in developing skeletal muscle cells which disappears after the muscle matures and becomes innervated by a nerve. Thus, myoD1 and myogenin are not usually found in normal skeletal muscle and serve as a useful immunohistochemical marker of rhabdomyosarcoma. Early manifestation can be misdiagnosed as a pseudotumor that is non responsive to steroid treatment.

Photomicrograph showing nodules of tumor cells separated by hyalinised fibrous septae (50×, HE stain). Inset: Discohesive large tumor cells with hyperchromatic nucleus and scant cytoplasm (200×, HE stain). The diagnosis was postauricular congenital alveolar rhabdomyosarcoma. Several different histological subtypes of rhabdomyosarcoma exist, each of which has different clinical and pathological characteristics. Prognosis and clinical behavior of the tumor is also partially dependent on histologic subtype. Multiple classification systems have been proposed for subclassifying these tumors. The most recent classification system, the “International Classification of Rhabdomyosarcoma,” was created by the Intergroup Rhabdomyosarcoma Study. This system attempts to combined elements of the previous systems and correlate these with prognosis based on tumor type.

Several additional subtypes of rhabdomyosarcoma exist that do not fit into the International Classification scheme. Pleomorphic rhabdomyosarcoma usually occurs in adults rather than children, and, thus, is not included in this system. Sclerosing rhabdomyosarcoma is a rare rhabdomyosarcoma subtype recently characterized by Folpe, et al.; it is not included in this system. Although botryoid and spindle cell rhabdomyosarcoma are classically considered as subtypes of embryonal rhabdomyosarcoma, they have more favorable clinical behavior and prognosis than classic embryonal rhabdomyosarcoma.

Treatment for rhabdomyosarcoma consists of chemotherapy, radiation therapy and sometimes surgery. Surgery to remove the tumor may be difficult or impossible depending on the location of the tumor. If there is no evidence of metastasis, surgery combined with chemotherapy and radiation offers the best prognosis. Patients whose tumors have not metastasized usually have a good chance for long-term survival, depending on the subtype of the tumor. St Jude's Children's Research Hospital reports that more than 70% of children diagnosed with localized rhabdomyosarcoma have long-term survival.

E. Sarcopenia

Sarcopenia (from the Greek meaning “poverty of flesh”) is the degenerative loss of skeletal muscle mass (0.5-1% loss per year after the age of 25), quality, and strength associated with aging. Sarcopenia is a component of the frailty syndrome. As of 2009, there was no generally accepted definition of sarcopenia in the medical literature.

Sarcopenia is characterized first by a muscle atrophy (a decrease in the size of the muscle), along with a reduction in muscle tissue “quality,” caused by such factors as replacement of muscle fibres with fat, an increase in fibrosis, changes in muscle metabolism, oxidative stress, and degeneration of the neuromuscular junction. Combined, these changes lead to progressive loss of muscle function and frailty.

Lack of exercise is currently thought to be a significant risk factor for sarcopenia. Not only muscle but the entire musculoskeletal system of muscle, neuromuscular responsiveness, endocrine function, vasocapillary access, tendon, joint, ligament, and bone, depends on regular and lifelong exercise to maintain integrity. Exercise and increases in activity have been shown to be beneficial in settings of sarcopenia, even in the very old. However, even highly trained athletes experience the effects of sarcopenia. Even Master class athletes who continue to train and compete throughout their adult life, exhibit a progressive loss of muscle mass and strength, and records in speed and strength events decline progressively after age 30.

Simple circumference measurement does not provide enough data to determine whether or not an individual is suffering from severe sarcopenia. Sarcopenia is also marked by a decrease in the circumference of distinct types of muscle fibers. Skeletal muscle has different fiber-types, which are characterized by expression of distinct myosin variants. During sarcopenia, there is a decrease in “type 2” fiber circumference (Type II), with little to no decrease in “type I” fiber circumference (Type I), and deinervated type 2 fibers are often converted to type 1 fibers by reinnervation by slow type 1 fiber motor nerves.

Satellite cells are small mononuclear cells that abut the muscle fiber. Satellite cells are normally activated upon injury or exercise. These cells then differentiate and fuse into the muscle fiber, helping to maintain its function. One theory is that sarcopenia is in part caused by a failure in satellite cell activation. Therefore, the ability to repair damaged muscles or respond to nutritional signals is impaired.

Extreme muscle loss is often a result of both diminishing anabolic signals, such as growth hormone and testosterone, and promotion of catabolic signals, such as pro-inflammatory cytokines.

Due to the lessened physical activity and increased longevity of industrialized populations, sarcopenia is emerging as a major health concern. Sarcopenia may progress to the extent that an older person may lose his or her ability to live independently. Furthermore, sarcopenia is an important independent predictor of disability in population-based studies, linked to poor balance, gait speed, falls, and fractures. Sarcopenia can be thought of as a muscular analog of osteoporosis, which is loss of bone, also caused by inactivity and counteracted by exercise. The combination of osteoporosis and sarcopenia results in the significant frailty often seen in the elderly population.

Exercise has been considered of great interest in treatment of sarcopenia. There are several reports showing increased ability and capacity of skeletal muscle to synthesize proteins in response to short term resistance exercise. Also, it has been reported exercise can improve physical performance (strength and mobility) in elderly subjects. However, there is insufficient research demonstrating such findings in long term.

Currently, there are no agents approved for treatment of sarcopenia. Possible therapeutic strategies include use of testosterone or anabolic steroids, though long term use of these agents is controversial in men given concerns of prostate symptoms, and essentially contraindicated in women, given concerns of virilization. Recent experimental results have shown testosterone treatments may induce adverse cardiovascular events. Other approved medications have been shown to have little to no effect in this setting, including agents such DHEA and human growth hormone. New therapies in clinical development hold great promise in this area, including the selective androgen receptor modulators (SARMs), as evidenced by recent studies. Nonsteriodal SARMs are of particular interest, given they exhibit significant selectivity between the anabolic effects of testosterone on muscle, but apparently with little to no androgenic effects such as prostate stimulation in men or virilization in women.

### V. GENE TRANSFER

In accordance with the present disclosure, non-muscle cells will be subject to gene transfer of the Myomixer gene, and optionally the Myomaker gene and/or a further gene of interest, e.g., a therapeutic gene. Gene transfer methods generally fall into two general categories: viral and non-viral. The suitability of these methods will be determined by the particular constraints of the subject matter, such as the size and structure of the genes being transferred, and the type of cell into which the genetic material is to be delivered. Those of skill in the art can make appropriate selections from the myriad of different systems that are commonly used, many of which commercially available. The following is a general discussion of both types of methods.

A. Viral Transformation

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

Adenoviral Vectors.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present disclosure comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a ψ sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The iv sequence is required for the packaging of the adenoviral genome.

A common approach for generating an adenoviruses for use as a gene transfer vector is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present disclosure it may be convenient to introduce the transforming construct at the position from which the E1-coding sequences have been removed. However, the position of insertion of the construct within the adenovirus sequences is not critical to the disclosure. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. Nos. 5,670,488; 5,932,210, each specifically incorporated herein by reference).

Retroviral Vectors.

In certain embodiments of the disclosure, the use of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present disclosure may be genetically modified in such a way that some of the structural, infectious genes of the native virus have been removed and replaced instead with a nucleic acid sequence to be delivered to a target cell (U.S. Pat. Nos. 5,858,744; 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present disclosure are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

Herpesviral Vectors.

Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufman et al., 1999), tumors (Yoon et al., 1999; Howard et al., 1999), lung (Kohut et al., 1998), neuronal (Garrido et al., 1999; Lachmann and Efstathiou, 1999), liver (Miytake et al., 1999; Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or alpha genes, Early (E) or beta genes and Late (L) or gamma genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. Nos. 5,879,934; 5,851,826, each specifically incorporated herein by reference in its entirety). One IE protein, Infected Cell Polypeptide 4 (ICP4), also known as alpha 4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B-cell malignancies (Suzuki et al., 1998), ovarian cancer (Wang et al., 1998) and Duchenne muscular dystrophy (Huard et al., 1997).

Adeno-Associated Viral Vectors.

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Lentiviral Vectors.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art; see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Other Viral Vectors.

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1999) and influenza A virus (Neumann et al., 1999) are contemplated for use in the present disclosure and may be selected according to the requisite properties of the target system.

Chimeric Viral Vectors.

Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1997; Caplen et al., 2000) and adenoviral/adeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

B. Non-Viral Transformation

Suitable methods for nucleic acid delivery for transformation of cells or tissues for use with the current disclosure are believed to include virtually any method by which a nucleic acid (e.g., DNA) can be introduced, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection:

In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present disclosure include the introduction of a nucleic acid by direct microinjection. Direct microinjection has been used to introduce nucleic acid constructs into Xenopus oocytes (Harland and Weintraub, 1985).

Electroporation.

In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 9217598, incorporated herein by reference. Other examples of species for which protoplast transformation has been described include barley (Lazerri, 1995), sorghum (Battraw and Hall, 1991), maize (Bhattacharjee et al., 1997), wheat (He et al., 1994) and tomato (Tsukada, 1989).

Calcium Phosphate.

In other embodiments of the present disclosure, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading.

Additional embodiments of the present disclosure include the introduction of a nucleic acid by direct sonic loading. LTK⁻ fibroblasts have been transfected with the thymidine kinase gene by sonication loading (Fechheimer et al., 1987).

Liposome-Mediated Transfection.

In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor Mediated Transfection:

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present disclosure can be specifically delivered into a target cell in a similar manner.

C. Recipient Cells

Non-muscle recipient cells may be of virtually any cell type, but in particular may be fibroblasts (abundant in skin, hair, etc.) or red blood cells (ideal as circulating and contact with muscle). Also contemplated are cells of the bone marrow and adipose stem cells, as these cells are also highly abundant. Additionally, one could use muscle precursor cells as they are the “normal” cell type that fuses to muscle.

### VI. THERAPEUTIC TREATMENTS

The present disclosure contemplates the treatment of muscles disease using non-muscle cells expressing Myomixer (and Myomaker) to deliver therapeutic genes to muscle tissues. This is the first time, to the inventors' knowledge, that the technology to drive muscle-specific cell fusion using non-muscle cell carriers has been available.

In certain embodiments, it is envisioned that non-muscle cells expressing Myomixer/Myomaker and a therapeutic gene will be delivered to or near sites in subjects where muscle cells and/or tissues are lacking one or more gene products, leading to a disease phenotype. Alternatively, cells may be fused ex vivo, either using a histocompatible cell source or cells from a patient, and subsequently injected into, local to or regional to a disease site. In both instances, the fusion of non-muscle cells with muscle cells will deliver a therapeutic gene, and hence its gene product, to muscle cells lacking that gene product, thereby reversing or reducing the disease phenotype. One could also administer these transformed cells systemically. For instance, one could collect bone marrow cells and force expression of Myomixer/Myomaker and the therapeutic gene, and then transplant these cells into the patient (i.e., a bone marrow transplant).

A. CRISPR/Cas9

Of particular interest in the present disclosure is the use of a CRISPR/Cas9 system for correcting DMD and other genetic defects. CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

Repeats were first described in 1987 for the bacterium Escherichia coli. In 2000, similar clustered repeats were identified in additional bacteria and archaea and were termed Short Regularly Spaced Repeats (SRSR). SRSR were renamed CRISPR in 2002. A set of genes, some encoding putative nuclease or helicase proteins, were found to be associated with CRISPR repeats (the cas, or CRISPR-associated genes).

In 2005, three independent researchers showed that CRISPR spacers showed homology to several phage DNA and extrachromosomal DNA such as plasmids. This was an indication that the CRISPR/cas system could have a role in adaptive immunity in bacteria. Koonin and colleagues proposed that spacers serve as a template for RNA molecules, analogously to eukaryotic cells that use a system called RNA interference.

In 2007 Barrangou, Horvath (food industry scientists at Danisco) and others showed that they could alter the resistance of Streptococcus thermophilus to phage attack with spacer DNA. Doudna and Charpentier had independently been exploring CRISPR-associated proteins to learn how bacteria deploy spacers in their immune defenses. They jointly studied a simpler CRISPR system that relies on a protein called Cas9. They found that bacteria respond to an invading phage by transcribing spacers and palindromic DNA into a long RNA molecule that the cell then uses tracrRNA and Cas9 to cut it into pieces called crRNAs.

CRISPR was first shown to work as a genome engineering/editing tool in human cell culture by 2012 It has since been used in a wide range of organisms including bakers yeast (S. cerevisiae), zebra fish, nematodes (C. elegans), plants, mice, and several other organisms. Additionally CRISPR has been modified to make programmable transcription factors that allow scientists to target and activate or silence specific genes. Libraries of tens of thousands of guide RNAs are now available.

The first evidence that CRISPR can reverse disease symptoms in living animals was demonstrated in March 2014, when MIT researchers cured mice of a rare liver disorder. Since 2012, the CRISPR/Cas system has been used for gene editing (silencing, enhancing or changing specific genes) that even works in eukaryotes like mice and primates. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

See also U.S. Patent Publication 2016/0058889, which is incorporated by reference in its entirety.

B. Combination Therapies

The cells of the present disclosure may also be used in combination with one or more other “standard” therapies. When given in combination, these compositions one would generally be administered in a combined amount effective to achieve a reduction in one or more disease parameter. This process may involve contacting the subject with the both agents/therapies at the same time, e.g., using a single composition or pharmacological formulation that includes both agents, or by contacting the subject with two distinct compositions or formulations, at the same time. Alternatively, one treatment may precede or follow the other treatment by intervals ranging from minutes to weeks. One would generally ensure that a significant period of time did not expire between each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations. It also is conceivable that more than one administration of either therapy will be desired.

C. Pharmaceutical Compositions and Administration

Where therapeutic applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells, as well as for culturing cells for fusions. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intraperitoneal, or intravenous injection. Intramuscular injection will be preferred. Such compositions would normally be administered as pharmaceutically acceptable compositions.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15^{th} Ed., 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VIII. KITS

For use in the applications described herein, kits are also within the scope of the disclosure. Such kits can comprise a carrier, package or container that is compartmentalized to receive one or more containers such as vials, tubes, and the like, each of the container(s) comprising one of the separate elements to be used in the method, in particular, a Myomixer expression construct or a transformed cell comprising the same, optionally further include a Myomaker expression construct or a transformed cell comprising the same. The kit of the disclosure will typically comprise the container described above and one or more other containers comprising materials desirable from a commercial end user standpoint, including buffers, diluents, filters, needles, syringes, and package inserts with instructions for use. In addition, a label can be provided on the container to indicate that the composition is used for a specific therapeutic application, and can also indicate directions for either in vivo or in vitro use, such as those described above. Directions and or other information can also be included on an insert which is included with the kit.

### IX. EXAMPLES

The following examples are included to further illustrate various aspects of the disclosure. It should be appreciated by those of skill in the art that the techniques disclosed in the examples which follow represent techniques and/or compositions discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Generation of Lentivirus Library and Infection of C2C12 Cells.

Mouse GeCKOv2 CRISPR knockout pooled library was a gift from Feng Zhang (Addgene plasmid #1000000052) (Sanjana et al., 2014). For library lentivirus production, 1.8×107 Lenti-X 293T cells (Clontech, Cat. No. 632180) were placed in 15 cm tissue culture plates in 25 ml of DMEM (containing 1% penicillin/streptomycin, 10% FBS). Twelve hours later, transfection was performed using FuGENE6 (Promega, #E2692) with 16.9 μg of psPAX2 plasmid, 5.6 μg of pMD2.G plasmid and 22.5 μg of GeCKO plasmid library. Two days later, lentivirus-containing supernatants were filtered through a 0.45 μm filter and concentrated with Lenti-X Concentrator (Clontech, PT4421-2) following manufacturer's protocol. psPAX2 vector was a gift from Didier Trono (Addgene plasmid #12260). pMD2.G vector was a gift from Didier Trono (Addgene plasmid #12259). A portion of the freshly made lentivirus was used to measure the titer. Briefly, C2C12 cells were infected with different volumes of viral stock for two days, followed by another two days of puromycin selection (2 μg/ml). Cell survival was measured and referred to as the infection rate. Based on the virus titer, an adjusted amount of virus stock was used to infect C2C12 cells for a target MOI of 0.2, ensuring that most cells incorporated less than 2 lentivirus particles. Two days after infection, puromycin (Invitrogen) was added at a final concentration of 2 μg/ml to select infected cells for two days. After selection, the cells were switched to 2% horse serum in DMEM (containing 1% Penicillin/Streptomycin) to differentiate for one week with fresh medium changed at day 3 of differentiation. After differentiation, the myotubes were detached from the plates by incubation for 5 minutes with 0.00625% trypsin (diluted in PBS) in 37° C., and the plates were washed gently twice with PBS, from which the detached cells were combined and centrifuged at 300×g for 5 minutes. The myoblasts were then detached with 0.25% trypsin and washed twice with PBS to collect all the cells, which were spun down at 300×g for 5 minutes.

Genomic DNA Purification and Library Preparation for Readout Sequencing.

Genomic DNA from myoblasts and myotubes was purified using MasterPure™ DNA Purification Kit (MC85200) according to manufacturer's instructions. The inventors performed two successive PCR reactions (18 cycles for the first and 12 cycles for the second reaction) to add barcodes, stagger and Illumina sequencing primers using Herculase II Fusion DNA Polymerase (Agilent, #600675) (Sanjana et al., 2014). PCR products were purified by Agencourt AMPure XP magnetic beads and the DNA was analyzed by Qubit Fluorometric Quantitation. The purified PCR product was sequenced on a Hiseq 2500 with 75 bp single reads.

Sequencing Data Analysis

A reference list for GeCKO sgRNA sequences in the library was downloaded from Addgene (world-wide web at addgene.org/pooled-library/zhang-mouse-gecko-v2/) and demultiplexed FASTQ files were mapped to the reference file using Bowtie 2 requiring unique alignments with no mismatches. The sgRNA quantification and gene score calculation was done using sgRSEA (bchen4.github.io/sgrsea/).

Myomixer CRISPR sgRNA Knockout Experiments in Cultured Cells.

Two guides (#1: 5′-GCTGCTGCCTGTTGCCCGCC-3′ (SEQ ID NO: 32), and #2: 5′-AGGCCTCTCCAGAATCCGG-3′ (SEQ ID NO: 33)) that target the Myomixer ORF region were individually cloned into the lenti-CRISPR v2 vector, a gift from Feng Zhang (Addgene plasmid #52961) (Sanjana et al., 2014). Myomixer CRISPR-sgRNA and -control (empty lenti-CRISPR v2) lentivirus were made and used to infect C2C12 or primary myoblast cells followed by puromycin selection with a similar procedure performed for the lentivirus CRISPR library. After selection, the cells were switched to differentiation medium for one week followed by immunostaining, RNA, protein and genomic DNA extraction and analysis. To verify targeting of Myomixer in these cells, genomic DNA was amplified with the primers that amplify the targeting region (Forward: 5′-AGTTCAGGCTTCAGGTCAGAG-3′ (SEQ ID NO: 34), Reverse: 5′-GCTAGGGGAGTGGGAACTGT-3′ (SEQ ID NO: 35), PCR product size was 743 bp for non-targeted cells). PCR products were gel purified and cloned into pCRII Topo vector (ThermoFisher, K460001) and sequenced.

Quantitative Real-Time PCR (qPCR).

Total RNA was extracted from cultured cells or mouse tissue with TRIZOL (Invitrogen) and cDNA was synthesized using iScript™ Reverse Transcription Supermix (1708841). Gene expression was assessed using standard qPCR approaches with KAPA SYBR FAST qPCR Master Mix (KK4605). Analysis was performed on a StepOnePlus realtime PCR System (Applied Biosystems) with the following Sybr primers:

Myomixer-F:
(SEQ ID NO: 36)
5'-GTTAGAACTGGTGAGCAGGAG-3',
Myomixer-R:
(SEQ ID NO: 37)
5'-CCATCGGGAGCAATGGAA-3'.
Myomaker-F:
(SEQ ID NO: 38)
5'-CCTGCTGTCTCTCCCAAG-3',
Myomaker-R:
(SEQ ID NO: 39)
5'-AGAACCAGTGGGTCCCTAA-3'.
Myod1-F:
(SEQ ID NO: 40)
5'-CCACTCCGGGACATAGACTTG-3',
Myod1-R:
(SEQ ID NO: 41)
5'-AAAAGCGCAGGTCTGGTGAG-3'.
Myogenin-F:
(SEQ ID NO: 42)
5'-GAGACATCCCCCTATTTCTACCA-3',
Myogenin-R:
(SEQ ID NO: 43)
5'-GCTCAGTCCGCTCATAGCC-3'.
Myh8-F:
(SEQ ID NO: 44)
5'-GGAGAGGATTGAGGCCCAAAA-3',
Myh8-R:
(SEQ ID NO: 45)
5'-CACGGTCACTTTCCCTCCATC-3'.
18S-F:
(SEQ ID NO: 46)
5'-ACCGCAGCTAGGAATAATGGA-3',
18S-R:
(SEQ ID NO: 47)
5'-GCCTCAGTTCCGAAAACCA-3'.

The 2ΔΔCt method was used to analyze the relative changes in gene expression normalized against 18S rRNA expression.

Pax7+ and Twist2+ Muscle Progenitor Cell Isolation and RNA-Seq Analysis.

Twist2+ muscle progenitor cells are distinct from Pax7+ satellite cells and represent a population of myogenic progenitor cells that contributes to specific fiber types during muscle homeostasis and regeneration (Liu et al., 2017)). Twist2+ muscle progenitor cells were freshly isolated by FACS sorting from the skeletal muscle tissues of 8-week-old Twist2-CreERT2; R26-tdTO mice 10 days post-Tamoxifen injection. Pax7+ satellite cells were isolated by FACS sorting as the integrin-7-positive and CD45/CD31/Sca1-negative population. RNA quality was verified by the Agilent 2100. Bioanalyzer and RNA-seq was performed using Illumina HiSeq 2500 by UTSW Genomics and Microarray Core Facility.

Retroviral Expression.

Myomixer coding sequence oligonucleotides were synthesized by Integrated DNA Technologies and ligated to the pMXs-Puro Retroviral Vector (Cell Biolabs, # RTV-012) (Kitamura eta., 2003) with In-Fusion® HD Cloning Plus Kit (Clontech, #638910). After verifying the correct insertion by sequencing, the plasmids were amplified in Stable3 cells in an overnight culture, followed by plasmid preparation using NucleoBond® Xtra Maxi Columns (#740414.10). A retrovirus plasmid that expresses the N-terminus tagged Signal FLAG (SF) Myomaker was used to overexpress Myomaker (Millay et al., 2016). Ten micrograms of retroviral plasmid DNA were transfected using FuGENE 6 (Promega, #E2692) into Platinum-E cells (Cell Biolabs, #RV-101) which were plated on a 10 cm cell culture dish at a density of 5×10⁶ cells per dish 12 hours before transfection. Two days after transfection, viral medium was filtered through a 0.45 μm cellulose syringe filter, and mixed with polybrene (Sigma) at a final concentration of 6 μg/ml. Two days post-infection, the cells were washed twice with PBS. For fusion experiments, virus infected C2C12 and 10T1/2 cells were mixed (5×10⁴ C2C12 cells and 2×10⁵ fibroblasts per well) and plated onto a 6-well plate. Twelve hours after plating, the cells were switched to myoblast differentiation medium (2% horse serum in DMEM with 1% penicillin/streptomycin) for one week with a medium change at day 3 of differentiation.

Cell Cultures and Fluorescent Protein Labelling.

10T1/2 fibroblasts and C2C12 cells were maintained in 10% FBS with 1% penicillin/streptomycin in DMEM. Lentiviruses expressing GFP and retroviruses expressing Cherry were made as described above. The packaging vector for lentiviral GFP is pLove-GFP, a gift from Miguel Ramalho-Santos (Addgene plasmid #15949) (Blelloch et al., 2007) and retroviral Cherry is pMXs-Cherry (Cell Biolabs, #RTV-012). Twelve hours before infection, the cells were seeded in 10-cm dish at a 2×10⁶ density. Cells were infected for two days before used for experiments.

Membrane Fractionation.

Membrane fractionation was performed with the Mem-PER™ Plus Membrane Protein Extraction Kit (ThermoFisher, #89842). Briefly, the cells were suspended in PBS by scraping off the surface of the plate with a cell scraper. After centrifuging the cell suspension at 300×g for 5 minutes, the cell pellets were washed twice and permeabilized with constant mixing for 10 minutes at 4° C. The cytosol fraction (supernatant) was collected after 15 minutes centrifugation at 16,000×g at 4° C. The membrane fraction (pellets) were resuspended and solubilized at 4° C. for 30 minutes with constant mixing. The membrane fraction was collected as the supernatant after 16,000×g centrifugation for 15 minutes at 4° C. The protein samples were mixed with 4× Laemmli sample buffer and analyzed by western blot analysis.

Immunoprecipitation and Western Blot Analysis.

Cells were washed with ice-cold PBS and lysed in IP-A buffer (50 mM TRIS, 150 mM NaCl, 1 mM EDTA, 1% Triton X-100) supplemented with complete protease inhibitor (Sigma) and PhoSTOP phosphatase inhibitor (Sigma) for 15 minutes. Cell lysates were disrupted mechanically by passing them through 25 G 5/8 needles and then constantly mixed for 1 hour at 4° C. Lysates were then centrifuged at 20,817×g for 15 minutes and the pellet was discarded. 10% of total lysate was used as input and the remaining volume was used for co-immunoprecipitation. Anti-FLAG M2 affinity resin (Sigma, A220) was equilibrated in IP-A buffer according to manufacturer's instructions. 100 μl resin was used per sample. Co-immunoprecipitation was performed in a total volume of 2 ml at 4° C. under constant mixing for 12 hours. Resin was washed in high salt (700 mM NaCl) and elution was performed with FLAG peptide (Sigma, F390) at a final concentration of 200 μg/mL for 6 hours at room temperature under constant mixing. SDS-PAGE was performed as described below.

Protein was isolated from cells or tissue using RIPA buffer (Sigma, R0278). Protein concentrations were determined using BCA Protein Assay Reagent (ThermoFisher Scientific, 23225) followed by measurement with NanoDrop. Protein samples were mixed with 4× Laemmli sample buffer (BIO-RAD, #161-0747) and 20-40 μg protein was loaded and separated by Mini-PROTEAN® TGX™ Precast Gels and transferred to a polyvinylidene fluoride (PVDF) membrane (Millipore), blocked in 5% fat-free milk for 1 hour at room temperature, and then incubated with the following primary antibodies diluted in 5% milk overnight at 4° C.: Gapdh (Thermofisher, MA5-15738), N-Cadherin (Santa Cruz Biotechnology, sc7939), Insulin Receptor β (Cell Signaling Technology, #3020), EGF Receptor (Cell Signaling Technology, #2646), Tubulin (Sigma, T-6199), Myomixer (R&D Systems, #AF4580), FLAG (Sigma, F3165), Myosin (Sigma, M4273). The HRP-conjugated secondary antibodies: Donkey anti-sheep IgG-HRP (Santa Cruz Biotechnology, sc-2473), Goat Anti-Mouse IgG (H+L)-HRP Conjugate (BIO-RAD, #170-6516) and Goat Anti-Rabbit IgG (H+L)-HRP Conjugate (BIO-RAD, #170-6515) were diluted at 1:5,000. Immunodetection was performed using Western Blotting Luminol Reagent (Santa Cruz Biotechnology, sc2048).

Immunostaining.

Cells were fixed in 4% PFA/PBS for 10 minutes, and permeabilized with 0.2% Triton X-100 in PBS and blocked with 3% BSA/PBS for 1 hour at room temperature. Cells were incubated with primary antibody overnight at 4° C., followed by incubation with Alexa Fluor conjugated secondary antibody as indicated below. For live cell membrane-staining, cells were first washed with ice-cold PBS and blocked in 3% BSA/PBS for 15 min on ice. Primary antibody incubation was then performed using antibody against FLAG (M2 mouse anti-FLAG sigma, F3165 with a dilution of 1:500 in ice cold 1% BSA/PBS) on ice for 30 minutes. After primary antibody incubation, cells were washed with ice-cold PBS and fixed with 4% PFA/PBS at room temperature for 10 minutes. Cells were then incubated with Alexa-Fluor secondary antibody (ThermoFisher, A21422) for 30 minutes at room temperature. For Laminin staining, cells were permeabilized with 0.2% Triton X-100 in PBS and blocked with 3% BSA/PBS for 1 hour. Cells were incubated with primary antibody (rabbit anti-Laminin 1:500, 1 hour at room temperature), followed by incubation with secondary antibody (ThermoFisher, A21206) and Hoechst to counterstain the nucleus.

For immunostaining of muscle sections, 8 μm raw-embedded frozen sections were fixed with 4% PFA for 10 minutes, permeabilized with 0.01% Triton X-100 for 15 min, and then sections were incubated for 1 hour with M.O.M blocking kit (Vector Laboratories) to prevent background staining from endogenous mouse Ig. Sections were subsequently blocked in 10% normal donkey serum prepared in PBS and 0.01% Triton X-100 for 30 minutes and incubated with sheep antimyomixer (R&D, AP4580, 1:100 dilution) and mouse anti-MY32 primary antibodies overnight at 4° C. Primary antibodies were visualized with Alexa Fluor 568 and 488 (Life Technologies, 1:500). Nuclear counterstaining was performed with DAPI (Sigma, 1 μg/mL). The staining was visualized on a Zeiss LSM780 Confocal Microscope.

For histochemistry, mouse anti-skeletal myosin (MY-32 clone; 1:200 dilution) was used on paraffin sections in conjunction with Mouse-on-Mouse Blocking & Immunodection Kit and horseradish peroxidase (Vector Laboratories, Burlingame, Calif.). Bound myosin antibody was detected with diamonobenzedine chromagen (DAKO-Agilent, Carpinteria, Calif.) and slides counterstained with hematoxylin, according to previously described immunoperoxidase methods (Cianga et al., 1999; Borvak et al., 1998).

Generation of Myomixer Mutant Mice.

All animal procedures were approved by the Institutional Animal Care and Use Committee at the University of Texas Southwestern Medical Center. B6C3F1 mice were used as oocyte donors. Superovulated female B6C3F1 mice (6 weeks old) were mated to B6C3F1 stud males. Zygotes were harvested and kept in M16 medium (Brinster's medium for ovum culture with 100 U/ml penicillin and 50 mg/ml streptomycin) at 37° C. for 1 hour. Zygotes were transferred to M2 medium (M16 medium and 20 mM HEPES). Cas9 mRNA and Myomixer sgRNAs (#1 and #2) were injected into the pronucleus and cytoplasm. Injected zygotes were cultured in M16 medium for 1 hour at 37° C. and then transferred into the oviducts of pseudo-pregnant ICR female mice. E17.5 embryos were collected and processed for histology and gene expression assays. Tail genomic DNA was extracted and used for genotyping with primers that amplify the targeting region (Forward: 5′-AGTTCAGGCTTCAGGTCAGAG-3′ (SEQ ID NO: 48), Reverse: 5′-GCTAGGGGAGTGGGAACTGT-3′ (SEQ ID NO: 49), PCR product size is 743 bp for wild-type mouse).

Tissue Harvest and Preparation.

Mouse embryos for in-situ hybridization (ISH), immunohistochemistry (IHC), and routine histology were harvested from pregnant dams, grossly imaged, and immersion fixed in 4% paraformaldehyde. Subsequent paraffin processing, embedding, sectioning and H&E staining were performed by standard procedures (Shehan & Hrapchak, 1980; Woods & Ellis, 1996).

RNA In Situ Hybridization.

Probe template corresponding to the Myomixer ORF was cloned into pCRII-TOPO (Invitrogen, ThermoFisher, K460001). ³⁵S-labeled sense and antisense probes were generated by Sp6 and T7 RNA polymerases, respectively, from linearized cDNA templates by in vitro transcription using the Maxiscript kit (Ambion, Inc, Austin, Tex.). Radio-isotopic in-situ hybridization was performed as previously described (Shelton et a., 2000). Signal was revealed after 14 days auto-radiographic exposure.

Microscopy.

Gross embryo images were acquired on a Zeiss Stemi SV-11 stereo photo-dissection scope using an Optronics Macrofire digital CCD camera and PictureFrame 2.0 software (Optronics, Goleta, Calif.). Review and photography of all histologic preparations were carried out on a Nikon E600 photomicroscope equipped with bright field and incident angle dark field illumination (Nikon USA, Mellville, N.Y.). Images were captured at 1.25×, 4×, 10×(ISH), and 20×(IHC, H&E) objective magnifications using Nikon Elements 4.20.00 software.

### Example 2—Results

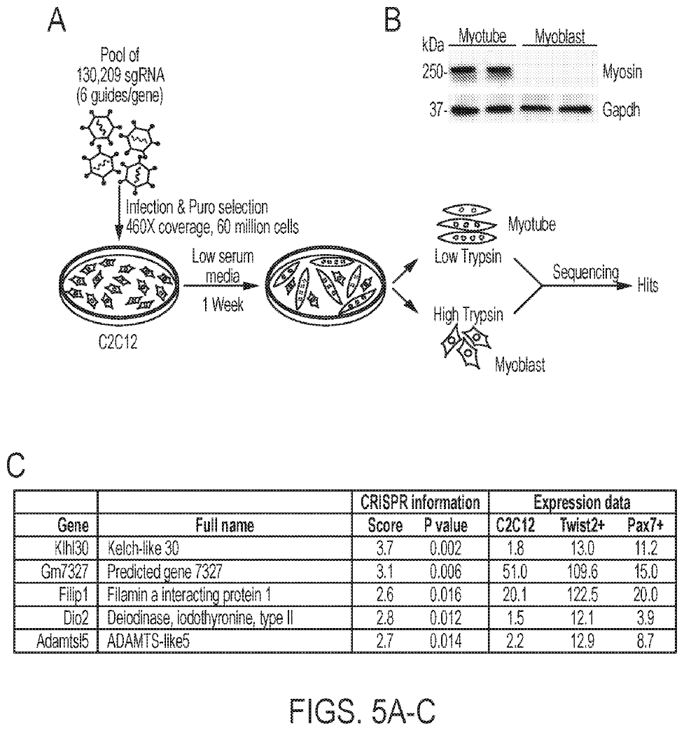

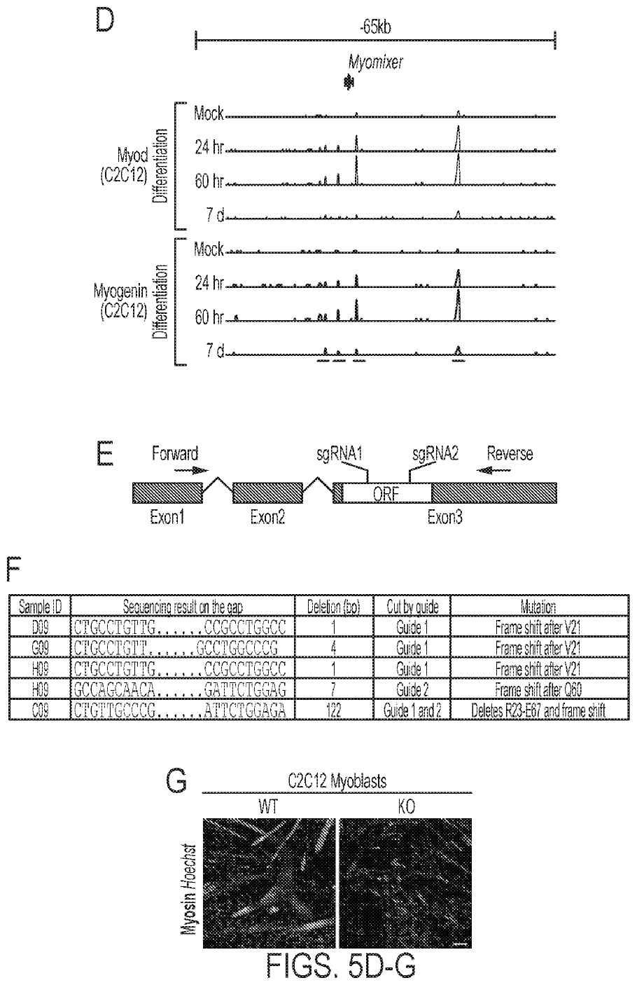

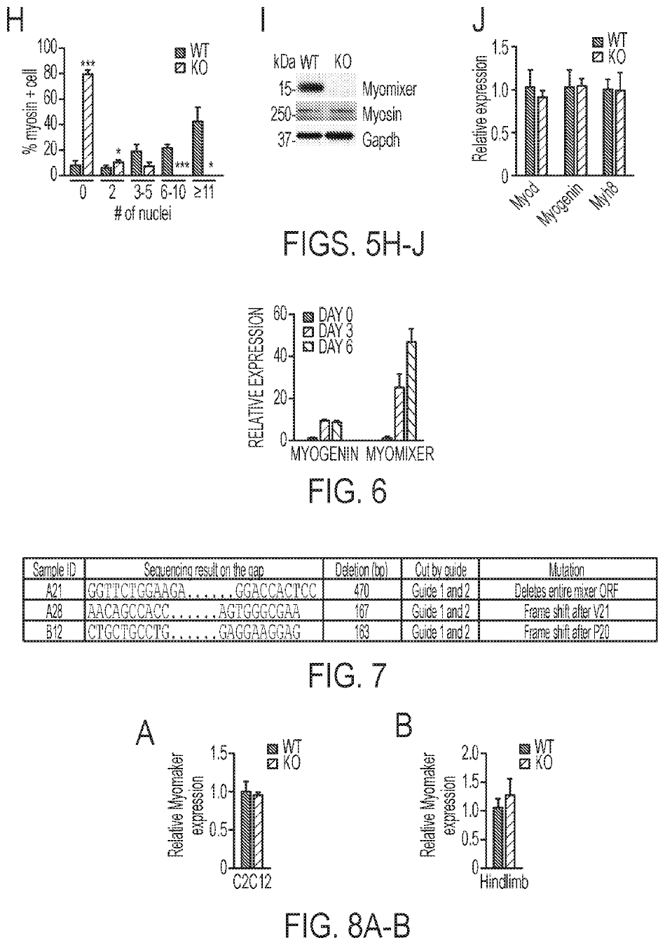

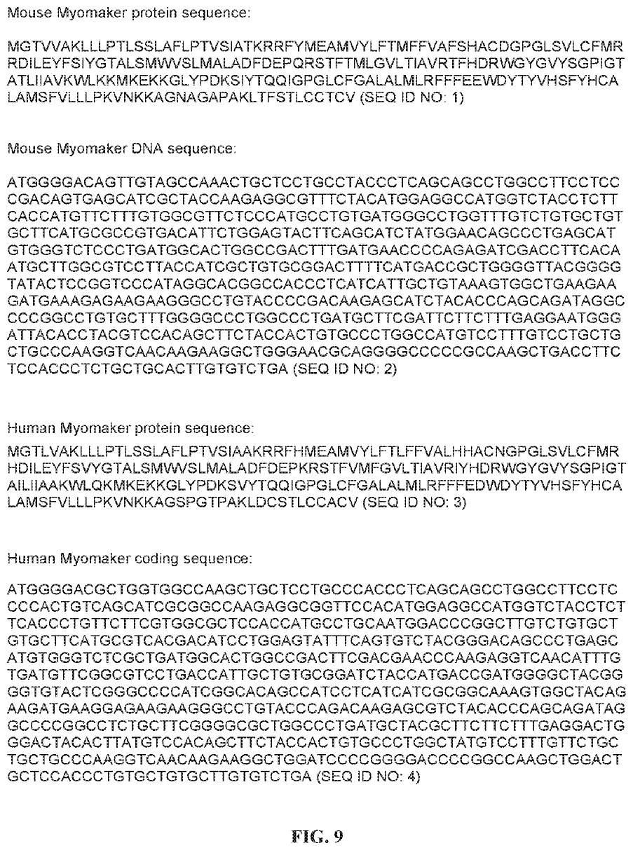

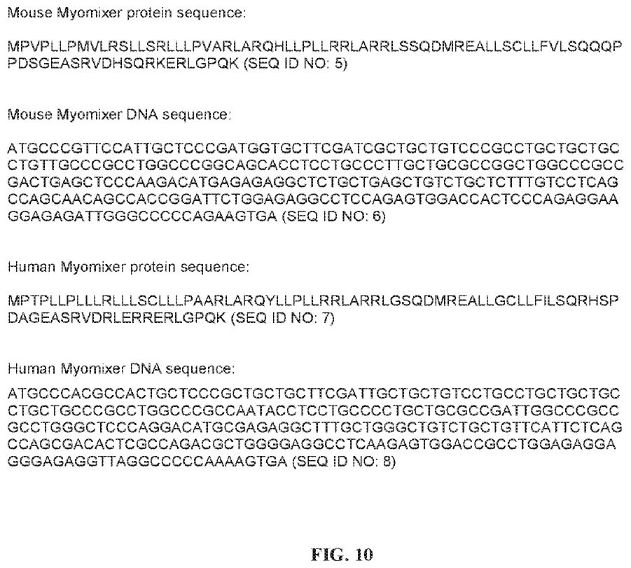

To identify new regulators of myogenesis, the inventors performed a genome-wide CRISPR loss-of-function screen for genes required for differentiation and fusion of mouse C2C12 muscle cells, as schematized in FIG. 5A. In brief, they infected 60 million C2C12 myoblasts with a lentiviral library comprising a pool of 130,209 single guide (sg) RNAs and Cas9 for CRISPR gene editing (Sanajan et al., 2014). Lentivirus infection was performed at a multiplicity of infection to retain a 460-fold representation of the library. Following puromycin selection for two days, myoblast cultures were switched to differentiation medium (DM) for one week to promote myotube formation. The cultures were subjected to a brief exposure with low trypsin (0.00625%), which promoted the detachment of myotubes, leaving mono-nucleated myoblasts attached to the dishes. Subsequent treatment with 0.25% trypsin allowed release of myoblasts. As a control for separation of the myotube and myoblast populations, the inventors detected myosin heavy chain by western blot analysis, which was highly enriched in the myotube population (FIG. 5B).

sgRNA representation in myoblast and myotube populations was enumerated by highthroughput sequencing and genes targeted by multiple myoblast-enriched sgRNAs were scored based on their relative abundance. This analysis revealed numerous genes that were targeted by multiple independent CRISPR sgRNAs that were enriched in myoblasts. Because the inventors sought to identify genes specifically required for myoblast differentiation or fusion, they narrowed down this list by comparing these genes to transcripts that are up-regulated during differentiation of C2C12 myoblasts (Chen et al., 2006), as well as Pax7+ satellite cells and Twist2+ myogenic progenitors (Liu et al., 2017) (FIG. 5C). Five genes fulfilled these criteria (FIG. 5C). One novel gene on the list, annotated as Gm7325, was targeted by 3 independent sgRNAs in the initial screen and stood out as the most up-regulated gene of unknown function during differentiation of C2C12 myoblasts and Pax7+ and Twist2+ muscle progenitors (FIG. 5C). This gene was also associated with conserved peaks for genomic binding of the myogenic transcription factors, MyoD and Myogenin (FIG. 5D) (Yue et al., 2014), and was identified as a putative target of MyoD (Fong et al., 2012).

There is a single publication describing Gm7325 as a gene expressed in embryonic stem (ES) cells and germ cells, but the function was not elucidated (Chen et a., 2005). Here, the inventors show that Gm7325 is required for myoblast fusion and promotes the mixing of cell membranes; thus, they have named this protein Myomixer. In the experiments that follow, the inventors demonstrate the key role of this protein in the control of myoblast fusion and muscle formation.

The mouse Myomixer gene spans three exons with the open reading frame (ORF) in exon 3 (FIG. 5E). To confirm the essentiality of Myomixer for myoblast fusion, the inventors disrupted the gene in C2C12 cells and mouse primary myoblasts with lentiviruses that expressed Cas9 and a pair of sgRNAs that were separated by 122 base pairs (bp) in the Myomixer ORF (FIG. 5E). Sequencing of genomic PCR fragments generated with primers flanking the sgRNA target sequences from these stably-selected cell cultures revealed numerous indel mutations that disrupted the Myomixer ORF (FIG. 5F).

Disruption of the Myomixer gene prevented fusion of C2C12 cells and primary mouse myoblasts, but did not affect expression of myosin heavy chain, a marker for differentiation (FIGS. 5G-5I, and FIGS. 1A-C). Quantification of the percentage of myonuclei in mono-versus multi-nucleated cells showed that ˜89% of myoblasts targeted with the Myomixer sgRNA pair were mono-nucleated after transfer to DM for seven days (FIG. 1B). The inventors never observed myotubes with more than five nuclei in the Myomixer knockout (KO) cultures, whereas ˜82% of myonuclei in wild type (WT) cultures were contained in myotubes with more than 5 nuclei (FIG. 1B). Western blot analysis confirmed the absence of Myomixer protein in the KO cultures (FIG. 5I and FIG. 1C). MyoD and myogenin were expressed normally in differentiated Myomixer KO cells, as was Myh8 (myosin heavy chain 8) (FIG. 5J), indicating a selective blockade to myoblast fusion independent of muscle differentiation.

The ORF of mouse Myomixer encodes a micropeptide that is 84 amino acids long (FIG. 1D). The protein was detected by western blot analysis with an antibody against amino acids 24-84 (FIG. 1C). Orthologs of Myomixer are conserved across diverse vertebrate species (FIG. 1D). Myomixer amino acid sequence is not annotated in fish or amphibians, presumably because ORFs smaller than 100 amino acids are not typically annotated. However, the inventors identified putative Myomixer orthologs in fish, frog and turtle genomes with conservation of several residues, especially many arginines and amphipathic residues (FIG. 1D). The proteins from the various species contain an N-terminal hydrophobic segment, followed by a positively charged helix and an adjacent hydrophobic helix. Myomixer proteins from mammals and marsupials also contain a unique C-terminal helix that is missing from other organisms (FIG. 1D).

Because the N-terminus contains an extended hydrophobic segment, the inventors postulated that it might serve as a membrane anchor. Indeed, immunostaining of intact C2C12 myoblasts expressing Myomixer with FLAG tag at the C-terminus revealed the presence of the protein on the cell surface (FIG. 1E). Consistent with these findings, fractionation of C2C12 myotubes into membrane and cytoplasmic fractions showed the localization of Myomixer to membranes (FIG. 1F). Similarly, in 293 cells infected with a Myomixer-expressing retrovirus, the inventors found that the protein was preferentially localized to the membrane (FIG. 1F).

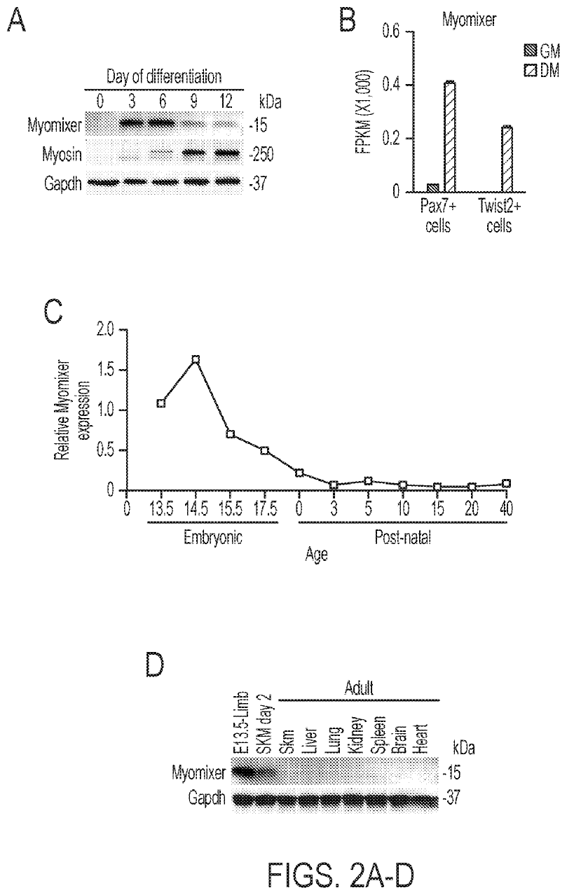

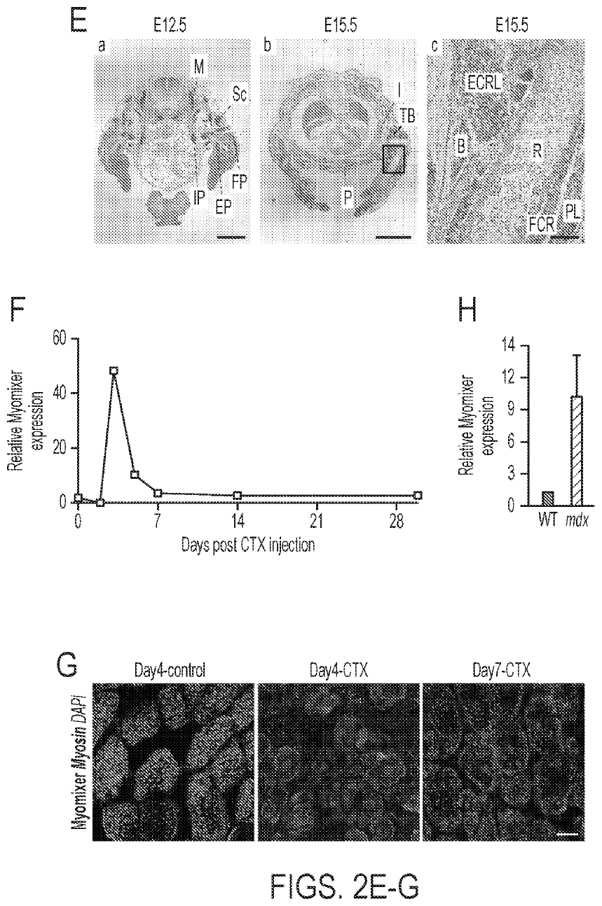

Myomixer protein expression was up-regulated during differentiation of C2C12 myoblasts and declined following myoblast fusion (FIG. 6A and FIG. 2A). Similarly, Myomixer was strongly up-regulated during differentiation of isolated Pax7+ satellite cells and Twist2+ adult muscle progenitor cells, which contribute to muscle regeneration (FIG. 2B). During pre- and post-natal muscle development, Myomixer transcripts showed a peak of expression at E14.5 and declined thereafter (FIG. 2C). Western blot analysis revealed the presence of the Myomixer protein in limb tissue from mouse embryos at E13.5 and in skeletal muscle at postnatal day 2, whereas the protein was undetectable in adult muscle of normal mice or in non-muscle tissues (FIG. 2D). In situ hybridizations of mouse embryo sections at E12.5 and E15.5 showed intense expression of Myomixer exclusively in developing skeletal muscles throughout the limbs and body wall (FIG. 2E). In response to cardiotoxin (CTX) injury of adult muscle, Myomixer expression was rapidly up-regulated, reaching a peak at day 3 post-injury and declining thereafter (FIG. 2F). Immunostaining of CTX treated muscle also showed robust expression of Myomixer in the myosin-positive muscle cells in the region of regeneration (FIG. 2G). Consistent with its potential role in muscle regeneration, Myomixer expression was up-regulated in muscle of mdx mice, which undergo muscle degeneration and regeneration during the progression of muscular dystrophy (FIG. 2H).

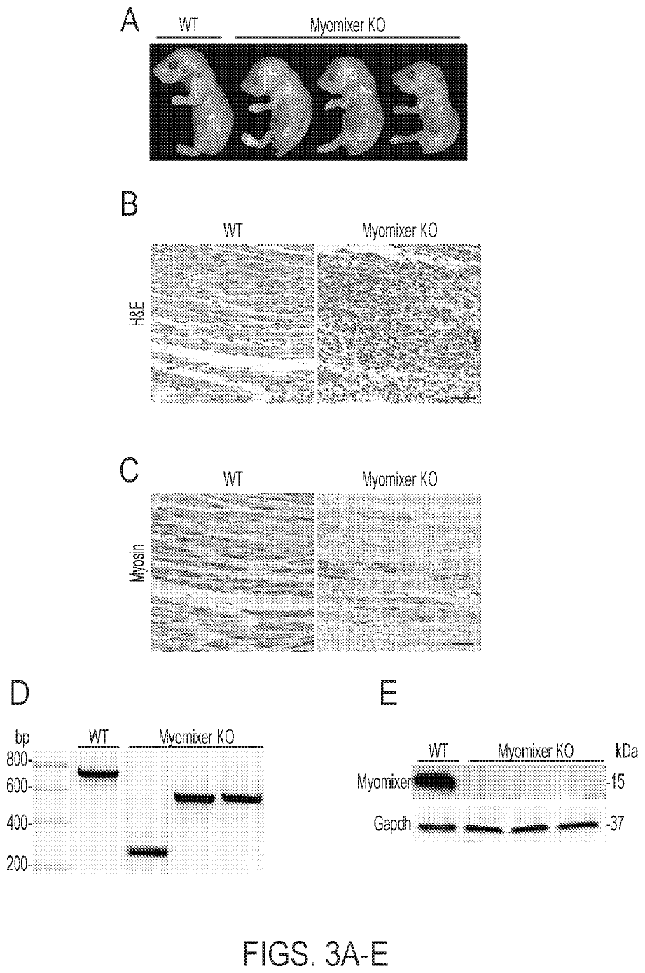

To assess the potential involvement of Myomixer in muscle formation in vivo, the inventors inactivated the gene during mouse embryogenesis by CRISPR-Cas9 mutagenesis using the same pair of sgRNAs that was shown to effectively target the gene in C2C12 cells and primary myoblasts. Fertilized zygotes were injected with Cas9 mRNA and sgRNAs, followed by implantation into pseudo-pregnant female mice. Embryos were harvested at embryonic day 17.5 (E17.5) and analyzed. From 65 embryos analyzed, the inventors obtained 9 motionless embryos that appeared to lack skeletal muscle and were nearly transparent such that internal organs and bones were apparent (FIG. 3A).

Histological sections through limb, body wall, and diaphragm musculature revealed an absence of multi-nucleated myofibers in Myomixer KO embryos (FIG. 3B). Instead, presumptive muscle-forming regions were populated by mono-nucleated cells that stained for myosin expression (FIG. 3C). Tissues other than muscle appeared normal in these embryos. The cutting sites of the two sgRNAs were 122 base pairs apart in the third exon of the Myomixer gene. PCR with primers amplifying this region showed mutant mice with deletions that ranged from 107-470 bp, reflecting different indels (FIG. 3D and FIG. 73). Western blot of hindlimbs confirmed the absence of Myomixer protein in the KO embryos (FIG. 3E).

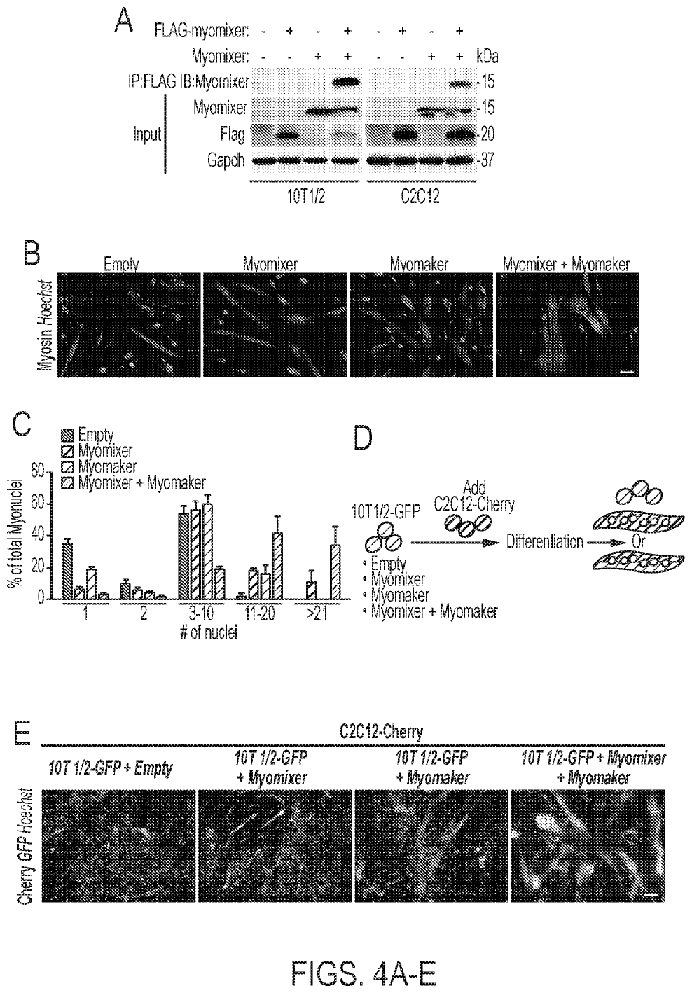

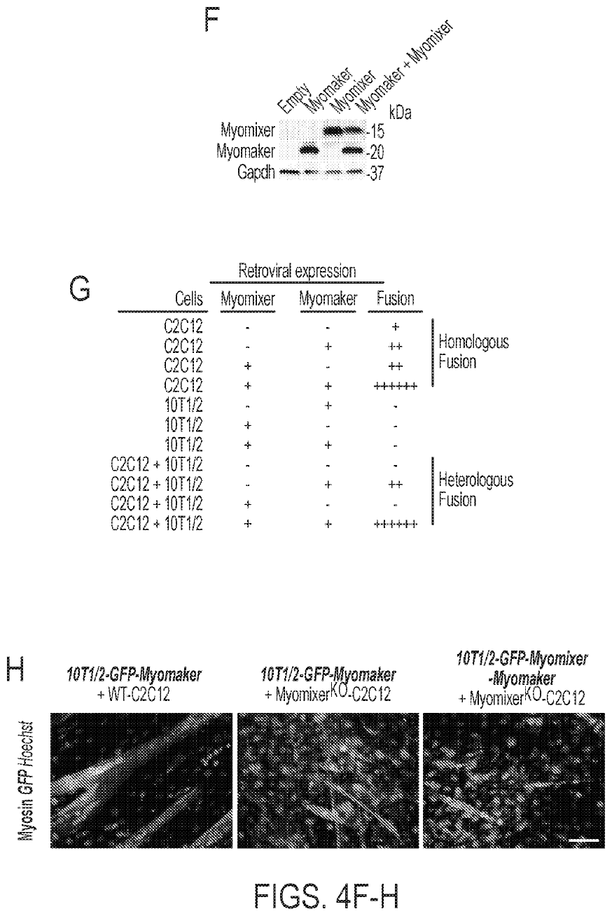

The muscle phenotype of Myomixer mutant mice is reminiscent of mice lacking Myomaker, a fusogenic muscle protein (Millay et al., 2016; 2013), suggesting a functional interaction between these two regulators of myoblast fusion. Indeed, in co-immunoprecipitation assays, FLAG-tagged Myomaker co-immunoprecipitated with Myomixer in 10T1/2 fibroblasts and differentiated C2C12 cells (FIG. 4A).

To further assess the functional interaction between Myomixer and Myomaker in myoblast fusion, the inventors retrovirally expressed each protein in C2C12 myoblasts and monitored myotube formation. As shown in FIG. 4B, Myomixer markedly enhanced the fusion of C2C12 cells. Moreover, when Myomixer and Myomaker were over-expressed together in C2C12 cells, they promoted the formation of massive multi-nucleated myotubes, indicative of their synergistic activity (FIG. 4B and FIG. 4C).

Myomaker can induce fusion of 10T1/2 fibroblasts with myoblasts (Millay et al., 2016; 2013). To test if Myomixer can synergize with Myomaker in promoting heterologous cell fusion, the inventors infected GFP-labelled 10T1/2 cells with Myomaker and Myomixer retroviruses (FIG. 4D). Remarkably, expression of Myomixer and Myomaker together in 10T1/2 cells induced dramatic fusion to C2C12 myotubes such that only a few mono-nucleated GFP-labeled fibroblasts remained in the cultures (FIG. 4E). Of note, although Myomaker-expressing 10T1/2 cells fused with C2C12 cells, Myomixer expressing 10T1/2 cells did not fuse (FIG. 4E). In addition, forced expression of Myomixer or Myomaker together, whether expressed in cis in the same cells or in trans in separate cells, did not induce fusion of 10T1/2 cells alone (data not shown). Western blot confirmed expression of Myomaker and Myomixer in the appropriate cells (FIG. 4F). The inventors observed no effect of either protein on the level of expression of the other, indicating that their synergy does not reflect an effect on stability of either protein. A summary of the effects of Myomixer and Myomaker on fusion is shown in FIG. 4G.

To test the functional dependency of Myomaker on Myomixer for cell fusion, the inventors mixed Myomaker-expressing 10T1/2 cells with WT or Myomixer-KO C2C12 myoblasts, and switched them to DM for one week, after which they stained for myosin. Strikingly, although Myomaker-expressing 10T1/2 cells fused with WT C2C12 cells, they were unable to fuse with Myomixer KO C2C12 cells (FIG. 4H). This implies that Myomaker relies on Myomixer for cell fusion in trans. Indeed, Myomaker was expressed normally in Myomixer KO myoblasts and embryos, suggesting a dependency of Myomaker on Myomixer for normal muscle fusion and development (FIG. 8).

The requirement of Myomixer for fusion of myoblasts in vivo and in vitro, its ability to synergistically promote fusion together with Myomaker, and the physical and functional interaction between these proteins indicate that they govern the critical step of multi-nucleation of skeletal muscle. Whereas the combination of Myomixer and Myomaker dramatically stimulates fusion of fibroblasts to myoblasts, these proteins cannot induce fusion of fibroblasts alone (FIG. 4G). Thus, additional myogenic partners are likely to contribute to the specificity of myoblast fusion.

The inventors have not found Myomixer-related genes in Drosophila, which also lacks an obvious Myomaker ortholog, suggesting that the Myomixer-Myomaker protein partnership is an invention of higher vertebrates. Perhaps the formation of the large muscles of vertebrates requires this robust fusogenic machinery superimposed upon the basic cell-cell fusion events of simpler organisms such as Drosophila.

The small size of Myomixer places it in the category of micropeptides, characterized by unprocessed ORFs of less than 100 amino acids (Marquez-Medina et al., 2015). It is noteworthy, in this regard, that a majority of micropeptides identified to date are embedded in membranes (Anderson et al., 2015; 2016). The inventors note some striking similarities between Myomixer and the heart-specific micropeptide, DWORF (Dwarf open reading frame), which localizes to the sarcoplasmic reticulum of cardiomyocytes where it associates with the SERCA calcium ATPase and stimulates its activity (Nelson et al., 2016). The inventors speculate that the activities of many membrane proteins may be governed by association with micropeptides that are as yet unidentified.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### X. REFERENCES

The following references, to the extent that they provide exemplary procedural or other details supplementary to those set forth herein, are specifically incorporated herein by reference.
U.S. Pat. No. 4,683,202
U.S. Pat. No. 5,928,906
U.S. Pat. No. 4,659,774
U.S. Pat. No. 4,816,571
U.S. Pat. No. 5,141,813
U.S. Pat. No. 5,264,566
U.S. Pat. No. 4,959,463
U.S. Pat. No. 5,428,148
U.S. Pat. No. 5,554,744
U.S. Pat. No. 5,574,146
U.S. Pat. No. 5,602,244
U.S. Pat. No. 4,682,195
U.S. Pat. No. 4,946,778
U.S. Pat. No. 5,645,897
U.S. Pat. No. 5,705,629
U.S. Pat. No. 5,925,565
U.S. Pat. No. 5,935,819
U.S. Pat. No. 5,670,488
U.S. Pat. No. 5,932,210
U.S. Pat. No. 5,858,744
U.S. Pat. No. 5,739,018
U.S. Pat. No. 5,879,934
U.S. Pat. No. 5,851,826
U.S. Pat. No. 6,013,516
U.S. Pat. No. 5,994,136
U.S. Pat. No. 5,871,983
U.S. Pat. No. 5,994,624
U.S. Pat. No. 5,871,982
U.S. Pat. No. 5,981,274
U.S. Pat. No. 5,945,100
U.S. Pat. No. 5,780,448
U.S. Pat. No. 5,736,524
U.S. Pat. No. 5,702,932
U.S. Pat. No. 5,656,610
U.S. Pat. No. 5,589,466
U.S. Pat. No. 5,580,859
U.S. Pat. No. 5,610,042
U.S. Pat. No. 5,322,783
U.S. Pat. No. 5,563,055
U.S. Pat. No. 5,550,318
U.S. Pat. No. 5,538,877
U.S. Pat. No. 5,538,880
U.S. Pat. No. 5,302,523
U.S. Pat. No. 5,464,765
U.S. Pat. No. 4,684,611
U.S. Pat. No. 4,952,500
U.S. Pat. No. 5,384,253
U.S. Pat. No. 5,789,215
Abmayr and Pavlath, Development, 139:641-656, 2012.
Almendro et al., J. Immunol., 157(12):5411-5421, 1996.
Anderson et al., Cell 160, 595-606, 2015.
Anderson et al., Sci Signal 9, [INSERT], 2016.
Ausubel et al., In: Current Protocols in Molecular Biology, John, Wiley & Sons, Inc, New York, 1994.
Barany and Merrifield, In: The Peptides, Gross and Meienhofer (Eds.), Academic Press, NY, 1-284, 1979.
Bates, Mol. Biotechnol., 2(2):135-145, 1994.
Battraw and Hall, Theor. App. Genet., 82(2):161-168, 1991.
Bentzinger et al., Cold Spring Harb Perspect Biol, 4, 2012.
Berkes and Tapscott, Semin Cell Dev Biol, 16:585-595, 2005.
Bett et al., J. Virololgy, 67(10):5911-5921, 1993.
Bhattacharjee et al., J. Plant Bioch. Biotech., 6(2):69-73. 1997.
Bilbao et al., Transplant Proc., 31(1-2):792-793, 1999.
Blelloch et al., Cell Stem Cell 1, 245-247, 2007.
Borvak et al., Int Immunol 10, 1289-1298, 1998.
Brack and Rando, Cell Stem Cell 10, 504-514, 2012.
Buckingham, Curr Opin Genet Dev, 16:525-532, 2006.
Buckingham and. Rigby, Dev Cell 28, 225-238, 2014.
Caplen et al., Gene, 252(1-2):95-105, 2000.
Carbonelli et al., “FEMS Microbiol Lett. 177(1):75-82, 1999.
Chandler et al., Proc. Natl. Acad. Sci. USA, 94(8):3596-601, 1997.
Charrasse et al., J Cell Biol, 158:953-965, 2002.
Charrasse et al., Mol Biol Cell, 18:1734-1743, 2007.
Chen and Okayama, Mol. Cell. Biol. 7:2745-2752, 1987.
Chen and Olson, Science 308, 369-373, 2005.
Chen and Olson, Dev Cell, 1:705-715, 2001.
Chen et al., Acta Bioch Bioph Sin 37, 789-796, 2005.
Chen et al., Bmc Cell Biol 7, [INSERT], 2006.
Chillon et al., J Virol., 73(3):2537-2540, 1999.
Christou et al., Proc. Natl. Acad. Sci. USA, 84(12):3962-3966, 1987.
Cianga et al., Eur J Immunol 29, 2515-2523, 1999.
Cocea, Biotechniques, 23(5):814-816, 1997.
Coffey et al., Science, 282(5392):1332-1334, 1999.
DeLuca et al., J. Virol., 56(2):558-570, 1985.
Demonbreun et al., Semin Cell Dev Biol 45, 48-56, 2015.
Derby et al., Hear Res., 134(1-2):1-8, 1999.
D'Halluin et al., Plant Cell, 4(12):1495-1505, 1992.
Doles and Olwin, Curr Opin Genet Dev 34, 24-28, 2015.
European Appln. EP 266 032
Fechheimer, et al., Proc Natl. Acad. Sci. USA, 84:8463-8467, 1987.
Feng et al., Nat. Biotechnol., 15(9):866-870, 1997.
Fisher et al., Virology, 217(1):11-22, 1996.
Fong et al., Dev Cell 22, 721-735, 2012.
Fraley et al., Proc. Natl. Acad. Sci. USA, 76:3348-3352, 1979.
Froehler et al., Nucleic Acids Res, 14; (13):5399-407.
Garrido et al., J. Neurovirol., 5(3):280-288, 1999.
Ghosh and Bachhawat, In: Liver Diseases, Targeted Diagnosis and Therapy Using Specific Receptors and Ligands, Wu and Wu (Eds.), Marcel Dekker, New York, 87-104, 1991.
Gnant et al., J. Natl. Cancer Inst., 91(20):1744-1750, 1999.
Gopal, Mol. Cell. Biol., 5:1188-1190, 1985.
Graham and Prevec Mol. Biotechnol., 3(3):207-220, 1995.
Graham and Van Der Eb, Virology 52:456-467, 1973.
Griffin et al., Dev Cell, 17:649-661, 2009.
Harland and Weintraub, J. Cell Biol., 101:1094-1099, 1985.
Hayashi et al., Neurosci. Lett., 267(1):37-40, 1999.
He et al., Plant Cell Reports, 14 (2-3):192-196, 1994.
Holzer et al., Virology, 253(1):107-114, 1999.
Hou and Lin, Plant Physiology, 111:166, 1996.
Howard et al., Ann. NY Acad Sci., 880:352-365, 1999.
Huard et al., Neuromuscul Disord., 7(5):299-313, 1997.
Imai et al., J. Virol., 72(5):4371-4378, 1998.
Kaeppler et al., Plant Cell Reports, 9:415-418, 1990.
Kaneda et al., Science, 243:375-378, 1989.
Kang and Krauss, Curr Opin Clin Nutr Metab Care, 13:243-248, 2010.
Kato et al., J. Biol. Chem., 266(6):3361-3364, 1991.
Kaufman et al., Surv. Ophthalmol., 43Suppl 1:S91-97, 1999.
Kitamura et al., Exp Hematol 31, 1007-1014, 2003.
Kohut et al., Am. J. Physiol., 275(6 Pt 1):L1089-94, 1998.
Kooby et al., FASEB J., 13(11):1325-1334, 1999.
Kraus et al., FEBS Lett., 428(3):165-170, 1998.
Krauss et al., Cold Spring Harb Perspect Biol 9, 2017.
Krisky et al., Gene Ther., 5(11):1517-1530, 1998a.
Krisky et al., Gene Ther., 5(12):1593-1603, 1998b.
Lachmann and Efstathiou, Clin. Sci. (Colch), 96(6):533-541, 1999.
Lareyre et al., J. Biol. Chem., 274(12):8282-8290, 1999.
Lazzeri, Methods Mol. Biol., 49:95-106, 1995.
Lee et al., Korean J. Genet., 11(2):65-72, 1989.
Lee et al., J. Auton. Nerv. Syst., 74(2-3):86-90, 1997.
Levenson et al., Human Gene Therapy, 9:1233-1236, 1998.
Liu et al., Nature Cell Biology, In press, 2017.
Lundstrom, J Recept. Signal Transduct. Res., 19(1-4):673-686, 1999.
Macejak and Sarnow, Nature, 353:90-94, 1991.
Marienfeld et al., Gene Ther., 6(6):1101-1113, 1999.
Marquez-Medina et al., Am J Clin Pathol 143, 889-894, 2015.
Merrifield, Science, 232(4748):341-347 1986.
Millay et al., Proc Natl Acad Sci USA 113, 2116-2121, 2016.
Millay et al., Nature 499, 301-305, 2013.
Moriuchi et al., Cancer Res., 58(24):5731-5737, 1998.
Morrison et al., J. Gen. Virol., 78(Pt 4):873-878, 1997.
Naldini et al., Proc. Natl. Acad. Sci. USA, 93(21):11382-11388, 1996.
Nelson et al., Science 351, 271-275, 2016.
Nicolau et al., Methods Enzymol., 149:157-176, 1987
Nicolau and Sene, Biochim. Biophys. Acta, 721:185-190, 1982.
Nomoto et al., Gene, 236(2):259-271, 1999.
Parks et al., J Virol., 71(4):3293-8, 1997.
PCT Appln. WO 94/09699
PCT Appln. WO 95/06128
PCT Appln. WO 92/17598
Pelletier and Sonenberg, Nature, 334:320-325, 1988.
Perales et al., Proc. Natl. Acad. Sci. USA, 91:4086-4090, 1994.
Potrykus et al., Mol. Gen. Genet., 199:183-188, 1985.
Potter et al., Proc. Natl. Acad. Sci. USA, 81:7161-7165, 1984.
Powell and Wright, PLoS Biol, 9:e1001216, 2011.
Omirulleh et al., Plant Mol. Biol., 21(3):415-28, 1993.
Oren-Suissa and Podbilewicz, Trends Cell Biol, 17:537-546, 2007.
Rabinovitch et al., Diabetes, 48(6):1223-1229, 1999.
Reddy et al., J Virol., 72(2):1394-1402, 1998.
Rhodes et al., Methods Mol. Biol., 55:121-131, 1995.
Rippe et al., “DNA-mediated gene transfer into adult rat hepatocytes in primary culture,” Mol. Cell Biol., 10:689-695, 1990.
Robbins and Ghivizzani, Pharmacol. Ther., 80(1):35-47, 1998.
Robbins et al., Proc. Natl. Acad. Sci. USA, 95(17):10182-10187 1998.
Robbins et al., Trends Biotechnol., 16(1):35-40, 1998.
Rochlin et al., Dev Biol, 341:66-83, 2010.
Sambrook et al., In: Molecular Cloning: A Laboratory Manual, Vol. 1, Cold Spring Harbor Laboratory Press, Cold Spring Harbor, N.Y., Ch. 7, 7.19-17.29, 1989.
Sambrook et al., In: Molecular cloning, Cold Spring Harbor Laboratory Press, Cold Spring Harbor, N Y, 2001.
Sanjana et al., Nature Methods 11, 783-784, 2014.
Schwander et al., Dev Cell, 4:673-685, 2003.
Shehan and Hrapchak, Theory and Practice of Histotechnology, 2nd ed., 1980.
Shelton et al., J Lipid Res 41, 532-537, 2000.
Shilagardi, et al., Science, 340:359-363, 2013.
Simionescu and Pavlath, Adv Exp Med Biol 713, 113-135, 2011.
Stewart and Young, “Solid Phase Peptide Synthesis”, 2d. ed., Pierce Chemical Co., 1984.
Suzuki et al., Oncogene, 17:853-865, 1998.
Suzuki et al., Biochem. Biophys. Res. Commun., 252(3):686-690, 1998.
Tam et al., J Am. Chem. Soc., 105:6442, 1983.
Tsukada et al., Plant Cell Physiol., 30(4)599-604, 1989.
Tsumaki et al., J. Biol. Chem., 273(36):22861-22864, 1998.
Tur-Kaspa et al., Mol. Cell Biol., 6:716-718, 1986.
Wagner et al., Science, 260:1510-1513, 1990.
Wang et al., Mol. Cell. Biol., 19:284-295, 1998.
Wang et al., Gynecol. Oncol., 71(2):278-287, 1998.
Wilson, J. Clin. Invest., 98(11):2435, 1996.
Wilson and Snell, Trends Cell Biol, 8:93-96, 1998.
Wong et al., Gene, 10:87-94, 1980.
Woods and Ellis, Laboratory Histopathology, a complete reference, 1996.
Wu et al., Biochem. Biophys. Res. Commun., 233(1):221-226, 1997.
Wu and Wu, J Biol. Chem., 262:4429-4432, 1987.
Yagami-Hiromasa, et al., Nature, 377:652-656, 1995.
Yamada et al., Proc. Natl. Acad. Sci. USA, 96(7):4078-4083, 1999.
Yeung et al., Gene Ther., 6(9):1536-1544, 1999.
Yin et al., Physiol Rev 93, 23-67, 2013.
Yoon et al., J. Gastrointest. Surg., 3(1):34-48, 1999.
Yue et al., Nature 515, 355-[INSERT], 2014.
Zhao-Emonet et al., Biochim. Biophys. Acta, 1442(2-3):109-119, 1998.
Zheng et al., J. Gen. Virol., 80(Pt 7):1735-1742, 1999.
Zhou et al., Exp. Hematol, 21:928-933, 1993.
Zufferey et al., Nat. Biotechnol., 15(9):871-875, 1997.

What is claimed is:

1. A cell transformed with an exogenous nucleic acid encoding a Myomixer polypeptide under the control of a promoter active in said cell.

2. The cell of claim 1, wherein said cell is a human cell.

3. The cell of claim 1, where said cell is a non-muscle cell.

4. The cell of claim 1, wherein said cell is a fibroblast, bone marrow cell or blood cell.

5. The cell of claim 1, wherein said exogenous nucleic acid is under the control of constitutive promoter or an inducible promoter.

6. The cell of claim 1, wherein said cell is further transformed with exogenous nucleic acid encoding a Myomaker polypeptide is under the control of a promoter active in said cell.

7. The cell of claim 1, wherein said exogenous nucleic acid is incorporated into a chromosome of said cell.

8. The cell of claim 1, wherein said exogenous nucleic acid is carried episomally by said cell.

9. The cell of claim 1, wherein said cell expresses a detectable marker and/or a selectable marker.

10. The cell of claim 1, wherein said cell is transformed to express a gene of interest other than Myomixer.

11. A method of preparing a non-muscle cell fusion partner comprising transferring into a non-muscle cell a nucleic acid encoding an exogenous Myomixer protein under the control of a promoter active in said cell.

12. The method of claim 11, wherein said cell is stably transformed.

13. The method of claim 11, wherein said cell is transiently transfected.

14. The method of claim 11, further comprising transferring into said cell a nucleic acid encoding or sufficient to produce a detectable marker.

15. The method of claim 11, wherein said exogenous nucleic acid is under the control of constitutive promoter or an inducible promoter.

16. The method of claim 11, wherein said cell is further transformed with exogenous nucleic acid encoding a Myomaker polypeptide is under the control of a promoter active in said cell.

17. The method of claim 11, wherein said cell is a human cell.

18. The method of claim 11, wherein said cell is a fibroblast, bone marrow cell or blood cell.

19. The method of claim 11, where said exogenous nucleic acid further encodes a selectable marker.

20. The method of claim 11, wherein said cell is transformed to express a gene of interest other than Myomixer.

21. A method of fusing a non-muscle cell to a muscle cell comprising:
(a) providing a non-muscle cell expressing (i) an exogenous Myomixer protein in said non-muscle cell and (ii) a Myomaker protein; and
(b) contacting said non-muscle cell with a muscle,
wherein said non-muscle cell expressing Myomixer protein will fuse with said muscle cell.

22. The method of claim 21, wherein said non-muscle cell is a human cell.

23. The method of claim 21, wherein said non-muscle cell is a fibroblast, bone marrow cell or blood cell.

24. The method of claim 21, wherein step (b) is performed in vitro.

25. The method of claim 21, wherein step (b) is performed in vivo.

26. The method of claim 21, wherein said non-muscle cell expresses a detectable marker or selectable marker.

27. The method of claim 21, wherein said non-muscle cell is further transformed with exogenous nucleic acid encoding a Myomaker polypeptide is under the control of a promoter active in said cell.

28. The method of claim 24, wherein said muscle cell is an isolated muscle cell.

29. The method of claim 21, wherein said muscle cell is located in intact muscle tissue.

30. The method of claim 21, wherein said muscle cell is a myoblast.

31. A method of delivering a gene of interest to a muscle cell comprising:
(a) providing a non-muscle cell expressing an exogenous Myomixer protein and a Myomaker protein, wherein said non-muscle cell further comprises a gene of interest; and
(b) contacting said non-muscle cell with a muscle cell,
wherein said non-muscle cell expressing Myomixer and Myomaker proteins will fuse with said muscle cell and deliver said gene of interest to said muscle cell.

32. The method of claim 31, wherein said non-muscle cell is a human cell.

33. The method of claim 31, wherein said non-muscle cell is a fibroblast, bone marrow cell or blood cell.

34. The method of claim 31, wherein step (b) is performed in vitro.

35. The method of claim 31, wherein step (b) is performed in vivo.

36. The method of claim 31, wherein said non-muscle cell expresses a detectable marker or a selectable marker.

37. The method of claim 31, wherein said non-muscle cell is further transformed with exogenous nucleic acid encoding a Myomaker polypeptide is under the control of a promoter active in said cell.

38. The method of claim 34, wherein said muscle cell is an isolated muscle cell.

39. The method of claim 31, wherein said muscle cell is located in intact muscle tissue.

40. The method of claim 31, wherein said muscle cell exhibits a pathologic phenotype, and said gene of interest correct said genotype.

41. The method of claim 40, wherein said pathologic phenotype is the underexpression or absence of a normal gene product.

42. The method of claim 40, wherein said pathologic phenotype is the expression of a defective gene product.

43. The method of claim 40, wherein said muscle cell has a pathologic phenotype associated with congenital myopathy, sarcopenia, amyotrophic lateral sclerosis, muscular dystrophy, Pompe disease or rhabdomyosarcoma.

44. The method of claim 40, wherein said non-muscle cell is delivered to an affected muscle tissue comprising said muscle in a subject.

45. The method of claim 44, wherein delivery is via intramuscular injection.

46. The method of claim 44, wherein delivery is repeated at least once.

47. The method of claim 44, wherein a secondary therapy is administered to said subject.

48. The method of claim 40, wherein said non-muscle cell is delivered to said muscle cell ex vivo and subsequently implanted in to a subject.

49. The method of claim 48, wherein said muscle cell is comprised in intact muscle tissue.

50. The method of claim 31, wherein said muscle cell is a myoblast.

51. An expression cassette comprising an exogenous nucleic acid encoding a Myomixer polypeptide under the control of a promoter active in eukaryotic cells.

52. The expression cassette of claim 51, wherein said exogenous nucleic acid is under the control of a constitutive promoter or an inducible promoter.

53. The expression cassette of claim 51, wherein said expression cassette further comprises an exogenous nucleic acid encoding a Myomaker polypeptide under the control of a promoter active in eukaryotic cells.

54. The expression cassette of claim 51, wherein said expression cassette encodes a detectable marker and/or a selectable marker.

55. The expression cassette of claim 51 or 53, wherein said expression cassette comprises an exogenous nuclei acid encoding a gene of interest other than Myomixer or Myomaker.

56. The expression cassette of claim 51, wherein said expression cassette is comprised in a replicable vector.

57. The expression cassette of claim 56, wherein said replicable vector is a viral vector.

58. The expression cassette of claim 57, wherein said viral vector is a retroviral vector, a lentiviral vector, an adenoviral vector or an adeno-associated viral vector.

59. The expression cassette of claim 56, wherein said replicable vector is a non-viral vector.

60. The expression cassette of claim 59, wherein said non-viral vector is formulated in a liposome or nanoparticle.

61. A method of correcting a genetic defect in a cell in a subject comprising:
(a) providing a non-muscle cell from said subject;
(b) introducing one or more expression cassettes into said non-muscle cell expressing (i) exogenous Myomixer and Myomaker proteins in said non-muscle cell and (ii) one or more therapeutic genes; and
(c) contacting said non-muscle cell with a muscle cell having a genetic defect,
wherein said non-muscle cell expressing Myomixer and Myomaker proteins will fuse with said muscle cell and deliver said therapeutic gene or genes to said muscle cell, thereby correcting said genetic defect.

62. The method of claim 61, wherein said non-muscle cell is a human cell.

63. The method of claim 61, wherein said non-muscle cell is a fibroblast, bone marrow cell or blood cell.

64. The method of claim 61, wherein steps (b) and/or (c) are performed in vitro or in vivo.

65. The method of claim 61, wherein step (b) is performed in vitro and step (c) is performed in vivo.

66. The method of claim 61, wherein said non-muscle cell expresses a detectable marker or a selectable marker.

67. The method of claim 61, wherein said therapeutic gene or genes comprises Cas9 and at least one therapeutic sgRNA.

68. The method of claim 61, wherein said muscle cell is an isolated muscle cell.

69. The method of claim 65, wherein said muscle cell is located in intact muscle tissue.

70. The method of claim 61, wherein said genetic defect is a Duchenne Muscular Dystrophy mutation.

71. The method of claim 61, wherein said genetic defect is congenital myopathy.

72. The method of claim 61, wherein said genetic defect is Pompe disease.

73. The method of claim 61, wherein said genetic defect is amyotrophic lateral sclerosis.

74. The method of claim 65, wherein said non-muscle cell is delivered to an affected muscle tissue comprising said muscle in a subject.

75. The method of claim 74, wherein delivery is via intramuscular injection.

76. The method of claim 74, wherein delivery is repeated at least once.

77. The method of claim 74, wherein a secondary therapy is administered to said subject.

78. The method of claim 65, wherein said non-muscle cell is contacted with said muscle cell ex vivo and subsequently implanted in to a subject.

79. The method of claim 65, wherein said muscle cell is comprised in intact muscle tissue.

80. The method of claim 61, wherein said muscle cell is a myoblast.

81. The method of claim 61, wherein said expression cassette or cassettes comprise a constitutive promoter or an inducible promoter.

82. The method of claim 61, wherein said expression cassette or cassettes encodes a detectable marker and/or a selectable marker.

83. The method of claim 61, wherein said expression cassette is comprised in a replicable vector.

84. The method of claim 83, wherein said replicable vector is a viral vector.

85. The method of claim 84, wherein said viral vector is a retroviral vector, a lentiviral vector, an adenoviral vector or an adeno-associated viral vector.

86. The method of claim 83, wherein said replicable vector is a non-viral vector.

87. The method of claim 86, wherein said non-viral vector is formulated in a liposome or nanoparticle.
